(19) United States
(12) Patent Application Publication
(10) Pub. No.: US 2023/0116978 A1
(43) Pub. Date: Apr. 20, 2023

(54) METHOD FOR LIBERATING INTRACELLULAR MATERIAL FROM PLANT CELLS

(71) Applicant: Deerland Enzymes, Inc., Kennesaw, GA (US)

(72) Inventor: John Deaton, Kennesaw, GA (US)

(73) Assignee: Deerland Enzymes, Inc., Kennesaw, GA (US)

(21) Appl. No.: 17/966,267

(22) Filed: Oct. 14, 2022

Related U.S. Application Data
(60) Provisional application No. 63/255,624, filed on Oct. 14, 2021.

(51) Int. Cl. A61K 36/21; A61P 39/06; A61P 29/00; A23L 33/105

(52) U.S. Cl. CPC A61K 36/21; A61P 39/06; A61P 29/00; A23L 33/105; A61K 2236/19; A23V 2002/00

(57) ABSTRACT

This disclosure relates to an enzymatic method including the steps of adding water to a reactor vessel with gentle mixing and minimal heating, adding osmotic protectant solution, adding plant material to the reactor, adding cell wall degrading enzymes at 0.1% wt/wt based on plant weight, heating the reactor now filled with plant material, osmotic protectant solution and enzyme blend solution to 30° C.-35° C., adding the enzyme mixture to the reactor and starting the timer for up to 2 hours, adding bulk density enhancer, further mixing the product, and drying the finished product containing protoplasts. The protoplast containing product may be provided in a dietary supplement for human use having antioxidant and anti-inflammatory properties.

This application claims the benefit of U.S. Provisional Application No. 63/255,624, filed on Oct. 14, 2021 which is hereby incorporated herein in its entirety for all purposes.

### TECHNICAL FIELD

The invention relates to a method of making protoplasts, intracellular material or individual chloroplasts obtained from plants after dissolution of the cell walls. In one embodiment the cell walls of plant cells are enzymatically degraded, leaving the phospholipid cell bilayer membrane intact as a unique protoplast.

### BACKGROUND

Current methods used to degrade the cell wall in plants are used to insert genetic material into the plant to ultimately obtain plants with improved properties. See, for example U.S. Pat. No. 5,508,184 to Negrutiu, et al., and references cited therein. No methods are optimized or used to obtain organelles from plant cells without the cell walls.

Furthermore, current techniques are only performed for plant breeding. Isolated protoplasts are also exploited in numerous miscellaneous studies involving membrane function, cell structure, synthesis of pharmaceutical products, and toxicological assessments.

If a method could be found by which protoplasts obtained from the enzymatic treatment of cell walls could be used for their health benefits without the need to use other organisms or create transgenic plants, this would represent a useful contribution to the art.

### SUMMARY

In an embodiment, a biochemical method is provided to release the protoplasts (altered forms of plant cells from which the cell wall has been partially or completely removed) or the plant cell without its cell wall to provide components that are needed to promote health. Certain plant cells are enzymatically treated to release cell contents and ultimately freeze dried to produce a powder. The dried protoplasts or a composition thereof may be used for its antioxidant properties and chaperone units contained therein.

An enzymatic method is described including the steps of adding water to a reactor vessel with gentle mixing and minimal heating, adding osmotic protectant solution, adding plant material (e.g., spinach) to the reactor, adding cell wall degrading enzymes at 0.1% wt/wt based on spinach weight, heating the reactor now filled with plant material, osmotic protectant solution and enzyme blend solution to 30° C.-35° C., adding the enzyme mixture to the reactor and starting the timer for up to 2 hours, adding bulk density enhancer, further mixing the product, and drying the finished product.

In a general embodiment, the following steps are followed.

Plant material is added to the reaction vessel.

Water is added to the reaction vessel in an amount approx. 50% of wt of plant material.

1% osmotic protectant solution is added to mix; 1% of osmotic protectant also wt/wt plant material.

Enzyme blend 0.1% wt/wt plant material is added; and a 30 min timer is started.

Gentle mixing is employed, and gentle heat not to exceed 37° C. for the duration of the preparation.

Optionally, at 30 mins—bulking agent may be added and mixed in.

The mixture is then rapidly cooled.

The product is freeze dried, then milled.

In another embodiment, a dietary supplement may include a protoplast enriched composition and a nutraceutically acceptable carrier.

### BRIEF DESCRIPTIONS OF THE DRAWINGS

FIG. 1 depicts the total antioxidant capacity (TAOC) of SOLARPLAST®, comparing the aqueous and DMSO-fractions, and the DMSO solvent control measured in Gallic Acid Equiv. (GAE) per mg, at various concentrations. The average±standard deviation of duplicate data points is shown for each dose of test product.

FIG. 2 depicts the total antioxidant capacity of freeze-dried Acai and Astaxanthin. The average±standard deviation of duplicate data points is shown for each dose of test product.

FIG. 3 depicts the total antioxidant capacity of Resveratrol. The average±standard deviation of duplicate data points is shown for each dose of test product.

FIG. 4 depicts the total antioxidant capacity of Chlorella. The average±standard deviation of duplicate data points is shown for each dose of test product.

FIG. 5 depicts the total antioxidant capacity of reduced glutathione. The average±standard deviation of duplicate data points is shown for each dose of test product.

FIG. 6 depicts the total antioxidant capacity of freeze-dried spinach compared to SOLARPLAST®. The average±standard deviation of duplicate data points is shown for each dose of test product.

FIG. 7 depicts the cellular antioxidant protection by SOLARPLAST®, comparing the aqueous fraction to the post-aqueous DMSO extract, with a DMSO control. Results are shown from the CAP-e bioassay. The percent inhibition of cellular oxidative damage is shown as the average±standard deviation of duplicate data points for each dose of test product.

FIG. 8 depicts the cellular antioxidant protection by SOLARPLAST®, comparing the aqueous fraction to the post-aqueous emulsified extract, with an emulsifier control. Results are shown from the CAP-e bioassay. The percent inhibition of cellular oxidative damage is shown as the average±standard deviation of duplicate data points for each dose of test product.

FIG. 9 depicts the cellular antioxidant protection by freeze-dried Acai and Astaxanthin: Results are shown from the CAP-e bioassay. The percent inhibition of cellular oxidative damage is shown as the average±standard deviation of duplicate data points for each dose of test product.

FIG. 10 depicts the cellular antioxidant protection by Resveratrol: Results are shown from the CAP-e bioassay. The percent inhibition of cellular oxidative damage is shown as the average±standard deviation of duplicate data points for each dose of test product.

FIG. 11 depicts the cellular antioxidant protection by Chlorella: Results are shown from the CAP-e bioassay. The percent inhibition of cellular oxidative damage is shown as the average±standard deviation of duplicate data points for each dose of test product.

FIG. 12 depicts the cellular antioxidant protection by reduced L-Glutathione: Results are shown from the CAP-e bioassay. The percent inhibition of cellular oxidative damage is shown as the average±standard deviation of duplicate data points for each dose of test product.

FIG. 13 depicts the cellular antioxidant protection by the aqueous fractions of freeze-dried spinach versus SOLARPLAST®: Results are shown from the CAP-e bioassay. The percent inhibition of cellular oxidative damage is shown as the average±standard deviation of duplicate data points for each dose of test product.

FIG. 14 depicts the cellular antioxidant protection by the post-aqueous emulsified extracts from SOLARPLAST® versus Acai: Results are shown from the CAP-e bioassay. The percent inhibition of cellular oxidative damage is shown as the average±standard deviation of duplicate data points for each dose of test product.

FIG. 15 depicts the effects on formation of ROS by human inflammatory polymorphonuclear (PMN) cells when exposed to SOLARPLAST®. The data is shown as percent change and as the average±standard deviation of triplicate data points for each dose.

FIG. 16 depicts the effects on formation of ROS by human inflammatory PMN cells when exposed to post-aqueous SOLARPLAST® in DMSO. The effects of the solvent DMSO are shown as comparison. The data is shown as the average±standard deviation of triplicate data points for each dose.

FIG. 17 depicts the effects on formation of ROS by human inflammatory PMN cells when exposed to post-aqueous emulsified SOLARPLAST®. The effects of the emulsifier are shown as comparisons. The data is shown as the average±standard deviation of triplicate data points for each dose.

FIG. 18 depicts the effects on formation of ROS by human inflammatory PMN cells when exposed to Freeze-dried Acai and Astaxanthin. The data is shown as the average±standard deviation of triplicate data points for each dose.

FIG. 19 depicts the effects on formation of ROS by human inflammatory PMN cells when exposed to Resveratrol. The data is shown as the average±standard deviation of triplicate data points for each dose.

FIG. 20 depicts the effects on formation of ROS by human inflammatory PMN cells when exposed to Chlorella. The data is shown as the average±standard deviation of triplicate data points for each dose.

FIG. 21 depicts the effects on formation of ROS by human inflammatory PMN cells when exposed to reduced L-Glutathione. The data is shown as the average±standard deviation of triplicate data points for each dose.

FIG. 22 depicts the effects on formation of ROS by human inflammatory PMN cells when exposed to Freeze-dried Spinach and SOLARPLAST®. The data is shown as the average±standard deviation of triplicate data points for each dose.

FIG. 23 depicts several representative antioxidant assays comparing SOLARPLAST® with Frozen spinach: A is a TOC assay, B is a FRAP assay, and C is a DPPH-TEAC assay, respectively.

FIG. 24 depicts NADP/NADPH quantification comparing SOLARPLAST® with Frozen spinach.

FIG. 25 depicts enzyme activity of various antioxidant enzymes present in spinach comparing SOLARPLAST® with Frozen spinach: A is a superoxide dismutase (SOD) assay, B is a peroxidase (POD) assay, C is a glutathione-S-transferase (GST) assay, D is a catalase (CAT) assay, E is a glutathione peroxidase (GPx) assay, and F is a monodehydroascorbate reductase (MDHAR) assay.

FIG. 26 depicts results of an assay for ROS/RNS testing placebo (PLB) and SOLARPLAST® following 45 days of supplementation by smokers. Two-way ANOVA indicated group by time interaction *p=0.031.

FIG. 27 depicts results of an assay for TNF-α testing placebo (PLB) and SOLARPLAST® following 45 days of supplementation by non-smokers. Two-way ANOVA indicated group by time interaction *p=0.024.

FIG. 28 depicts results of an assay for IL-4 testing placebo (PLB) and SOLARPLAST® following 45 days of supplementation by non-smokers.

FIG. 29 depicts results of an assay for IL-6 testing placebo (PLB) and SOLARPLAST® following 45 days of supplementation by non-smokers.

FIG. 30 depicts results of an assay TNF-α testing placebo (PLB) and SOLARPLAST® following 45 days of supplementation by smokers.

FIG. 31 depicts results of an assay for IL-4 testing placebo (PLB) and SOLARPLAST® following 45 days of supplementation by smokers.

FIG. 32 depicts results of an assay for IL-6 testing placebo (PLB) and SOLARPLAST® following 45 days of supplementation by smokers. Two-way ANOVA indicated group by time interaction *p=<0.001.

FIG. 33 is a photomicrograph of a protoplast (spheroplast) containing material prepared in accordance with an embodiment of the present invention, providing intact phospholipid bilayer cell membranes.

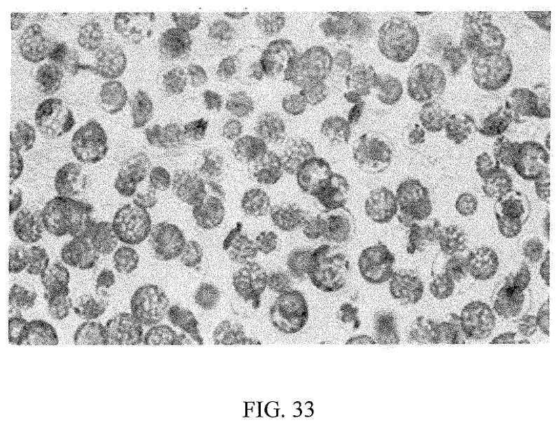

### DETAILED DESCRIPTION

The present disclosure provides a method for making protoplasts, intracellular material or individual chloroplasts obtained from plants after dissolution of the cell walls. The dried protoplasts or a composition thereof may be used for its antioxidant or anti-inflammatory properties.

Currently there are other antioxidant products in the market. However, many of these compounds need to be used in very large quantities. They are generally extracts from plants, and none are treated the way this method establishes, and therefore do not allow the inner organelles to be harvested without damaging the membranes and providing intact protoplasts.

As described herein, a protoplast containing, protoplast enriched composition (SOLARPLAST®, available from Deerland Probiotics and Enzymes, Kennesaw, Ga.) is plant material that has been harvested in very specific conditions to optimize enzymes, chaperones, and other nutrient components. The cell wall has been enzymatically degraded, leaving the phospholipid bilayer cell membrane intact. This preserves the light to energy converting activity of the organelles within the cell and also makes nutrients and minerals more bioavailable within the human body. The human body is able to easily break down and absorb all of the nutrients that plant cells contain naturally. SOLARPLAST® can also be described as a collection of light to energy converting organelles, mainly chloroplasts, encased in endogenous lipid bilayers, with high concentrations of antioxidant, energy, chaperone and fat blocker components.

The protoplast containing composition described herein provides the glutathione antioxidant recycling mechanism.

The most abundant antioxidant within all cells is glutathione. It is nearly ubiquitous in cells and has been shown to participate in a multitude of cellular functions. Although many of these functions are associated with protection against highly reactive molecules, including free radicals, maintenance of a suitable thiol redox is crucial for cellular homeostasis. Thiol redox is essential for regular cellular function such as signaling, metabolic, and transcriptional processes.

In one embodiment, the protoplast containing compositions may be used to protect the body from the stress caused by free radicals. The composition provides cleansing antioxidants that slow the cell damage that comes with many everyday activities and environmental conditions.

SOLARPLAST® proves to be a powerful antioxidant acting on all naturally occurring free radicals in a continuous manner, and also without producing harmful byproducts. It contains a unique mixture of antioxidant molecules that attacks all forms of oxidants and provides the components required to perpetuate the glutathione recycling mechanism leaving no harmful byproducts itself.

Report 157-001. Antioxidant and anti-inflammatory properties of a novel enzymatic digest of spinach: Comparison of fractions, and comparison to leading antioxidants on the market.

This study showed the effects of SOLARPLAST® in the following assays:

1. Total antioxidant capacity;

2. Cellular antioxidant protection (CAP-e bioassay); and

3. Effects on free radical (ROS) formation by inflammatory cells in human polymorphonuclear (PMN) cells.

This report discusses the tests conducted and the comparisons drawn on the aqueous and lipid fractions of a novel nutritional product in a selected panel of in vitro tests, focused on antioxidant and anti-inflammatory properties. As part of the testing, researchers compared results to 6 competing water-soluble natural products or extracts.

The following test products were compared in this project:

1. Spinach digest, aqueous fraction of SOLARPLAST®; 2. Spinach digest, post-aqueous fraction of SOLARPLAST® solubilized in DMSO; 3. Spinach digest, post-aqueous fraction of SOLARPLAST® in emulsifier; 4. DMSO control; 5. Emulsifier control. 6. Competing product 1: Acai berry extract; 7. Competing product 2: Astaxanthin (water-soluble); 8. Competing product 3: Resveratrol; 9. Competing product 4: Chlorella; 10. Competing product 5: Freeze-dried spinach; 11. Competing product 6: L-glutathione, reduced 97%.

For the cellular antioxidant protection assay, an additional test was performed to compare the lipid-soluble fractions of SOLARPLAST® and Acai, using an emulsification process.

Total antioxidant capacity was tested in the Folin-Ciocalteu assay (also known as the total phenolics assay). This assay makes use of the Folin-Ciocalteu reagent to measure antioxidants. The assay is performed by adding the Folin-Ciocalteu's phenol reagent to serial dilutions of extract, thoroughly mixing, and incubating for 5 minutes. Sodium carbonate is added, starting a chemical reaction producing a color. The reaction is allowed to continue for 30 minutes at 37° C. Optical absorbance is measured at 765 nm in a colorimetric plate reader. Gallic acid is used as a reference standard, and the data reported in Gallic Acid Equivalents per gram product.

Results showed that the aqueous fraction of SOLARPLAST® contained more antioxidants than the post-aqueous DMSO-fraction; the aqueous fraction of SOLARPLAST® contained more antioxidants than Astaxanthin, Chlorella, and freeze-dried spinach, and; Freeze-dried Acai, Resveratrol and reduced L-glutathione had more antioxidant capacity than SOLARPLAST®. Table 1 shows the comparative results.

TABLE 1
Comparison of antioxidant capacity.

| | Total antioxidant capacity (mg GAE per gram) |
|---|---|
| SolarPlast, aqueous | 4 |
| SolarPlast, DMSO | 1 |
| Freeze-dried Acai | 14 |
| Astaxanthin | 2 |
| Resveratrol | 62 |
| Chlorella | 1 |
| Freeze-dried Spinach | 2 |
| L-glutathione, reduced | 153 |

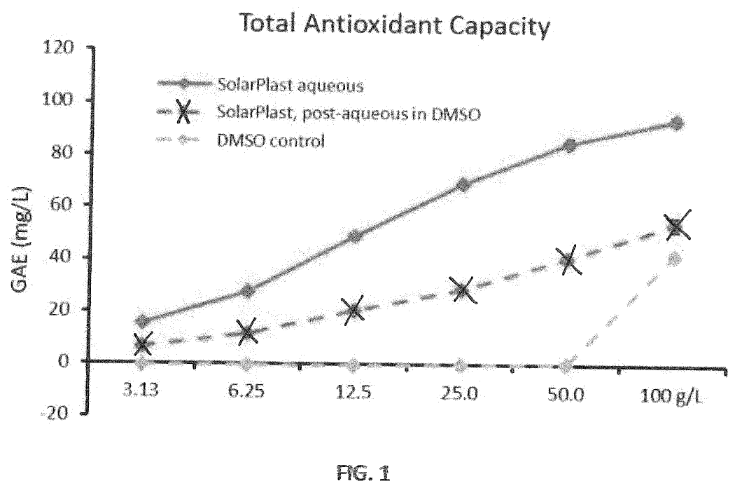

FIG. 1 describes the total antioxidant capacity of SOLARPLAST®, comparing the aqueous and DMSO-fractions, and the DMSO solvent control. The average±standard deviation of duplicate data points is shown for each dose of test product.

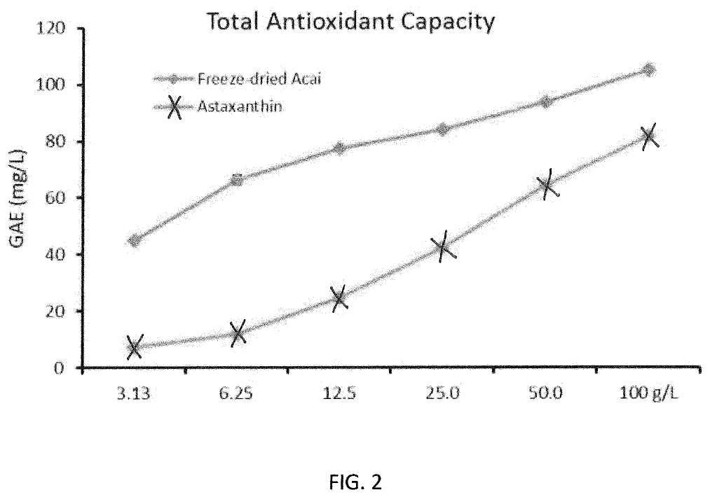

FIG. 2 describes the total antioxidant capacity of freeze-dried Acai and Astaxanthin. The average±standard deviation of duplicate data points is shown for each dose of test product.

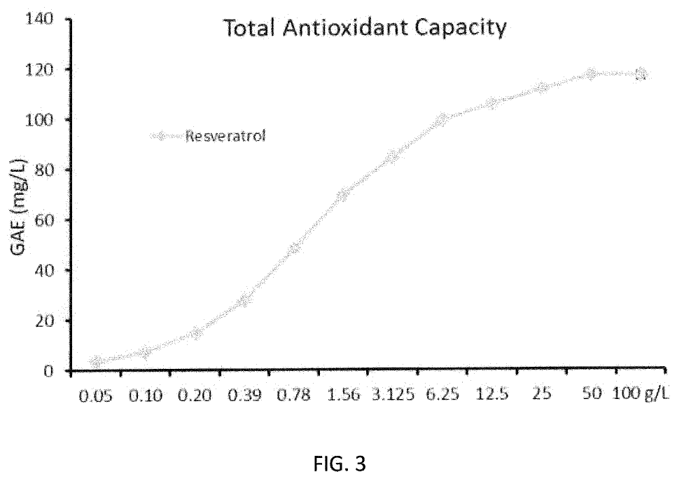

FIG. 3 describes the total antioxidant capacity of Resveratrol. The average±standard deviation of duplicate data points is shown for each dose of test product.

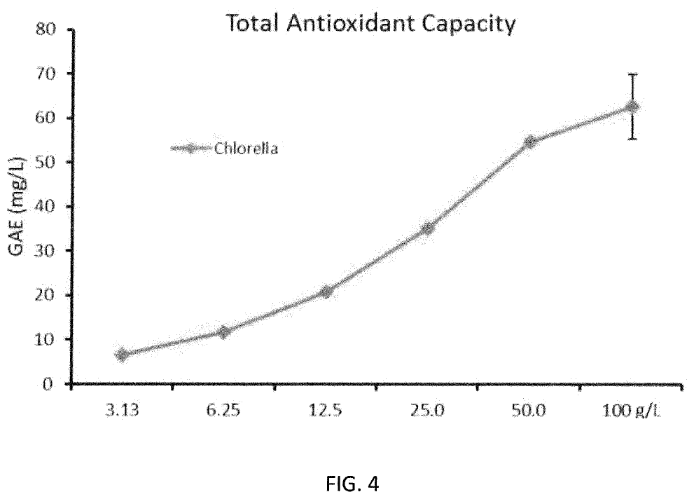

FIG. 4 describes the total antioxidant capacity of Chlorella. The average±standard deviation of duplicate data points is shown for each dose of test product.

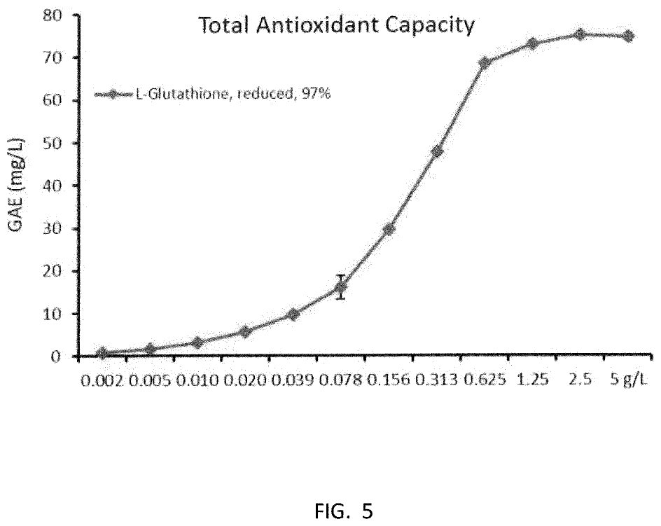

FIG. 5 describes the total antioxidant capacity of reduced glutathione. The average±standard deviation of duplicate data points is shown for each dose of test product.

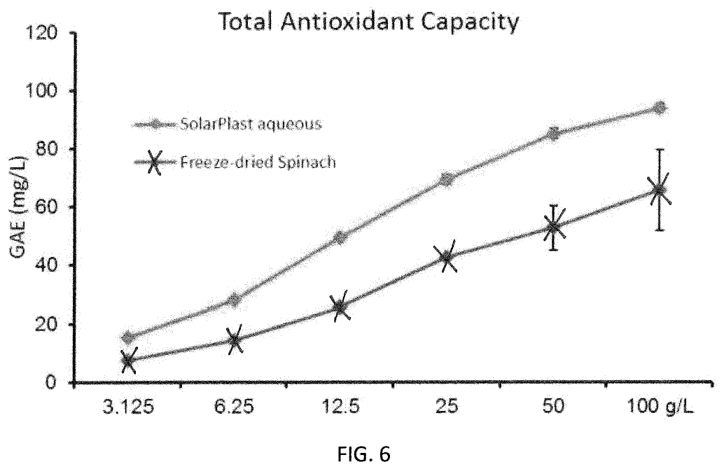

FIG. 6 describes the total antioxidant capacity of freeze-dried spinach compared to SOLARPLAST®. The average±standard deviation of duplicate data points is shown for each dose of test product.

Cell-based antioxidant protection was measured via a CAP-e bioassay. This allowed assessment of anti-oxidant potential in a method that is comparable to the Oxygen Radical Antioxidant Capacity (ORAC) test, but only allows measurement of antioxidants that are able to cross the lipid bilayer cell membrane, enter the cells, and provide biologically meaningful antioxidant protection under conditions of oxidative stress.

The CAP-e bioassay was specifically designed to work with natural products and ingredients.

The red blood cell (RBC) was used as the model cell type as it is an inert cell type, in contrast to other cell types such as PMN cells. The red blood cell-based assay was developed particularly to be able to assess antioxidants from complex natural products in a cell-based system, as well as help interpret subsequent data from more complex cellular models.

Freshly purified human RBC are washed repeatedly in physiological saline, and then exposed to the test products. During the incubation with a test product, any antioxidant compounds able to cross the cell membrane can enter the interior of the RBC. Then the RBC are washed to remove compounds that were not absorbed by the cells, and loaded with the DCF-DA dye, which turns fluorescent upon exposure to reactive oxygen species (ROS). Oxidation is triggered by addition of the peroxyl free radical generator AAPH. The fluorescence intensity is evaluated. The low fluorescence intensity of untreated control cells serves as a baseline, and RBC treated with AAPH alone serve as a positive control for maximum oxidative damage.

A reduced fluorescence intensity of RBC exposed to a test product and subsequently exposed to AAPH indicates that the test product contains antioxidants available to penetrate into the cells and protect these from oxidative damage.

Based on the low fluorescence of the untreated control wells, and the high fluorescence of the cell cultures exposed to oxidative damage, the fluorescence intensity in cell cultures treated with test products prior to exposure to oxidative stress is used to calculate the percent inhibition of cellular oxidative stress.

Results showed that the aqueous fraction of SOLARPLAST® provided more cellular antioxidant protection, i.e. contained a higher level of bioavailable antioxidants at the cellular level, than the postaqueous DMSO-fraction. The emulsified post-aqueous fraction of SOLARPLAST® also provided cellular antioxidant protection. The aqueous fraction of SOLARPLAST® provided similar cellular antioxidant protection as freeze-dried spinach. Freeze-dried Acai, and Resveratrol and L-glutathione (reduced) had more antioxidant capacity than SOLARPLAST®. For Astaxanthin, Chlorella, and the post-aqueous emulsified fraction of Acai, cellular antioxidant protection was evident, but the level of protection did not reach 50% (IC50). Since an IC50 value was not achieved, a CAP-e value could not be calculated. Table 2 shows the comparative results.

TABLE 2
Comparison of cellular antioxadent protection.

| | Cellular antioxidant Protection (μM GA/gram) |
|---|---|
| SolarPlast, aqueous | 13 |
| SolarPlast, | N/A* |
| post-aqueous in DMSO | |
| SolarPlast, | 4.8 |
| post-aqueous, emulsified | |
| Freeze-dried Acai | 59 |
| Freeze-dried Acai, | N/A* |
| post-aqueous, emulsified | |
| Astaxanthin | N/A* |
| Resveratrol | 235 |
| Chlorella | N/A* |
| Freeze-dried Spinach | 12 |
| L-glutathione, reduced | 619 |

*Cellular antioxidant protection was evident, but the level of protection did not reach 50% (IC50). Since an IC50 value was not achieved, a CAP-e value could not be calculated.

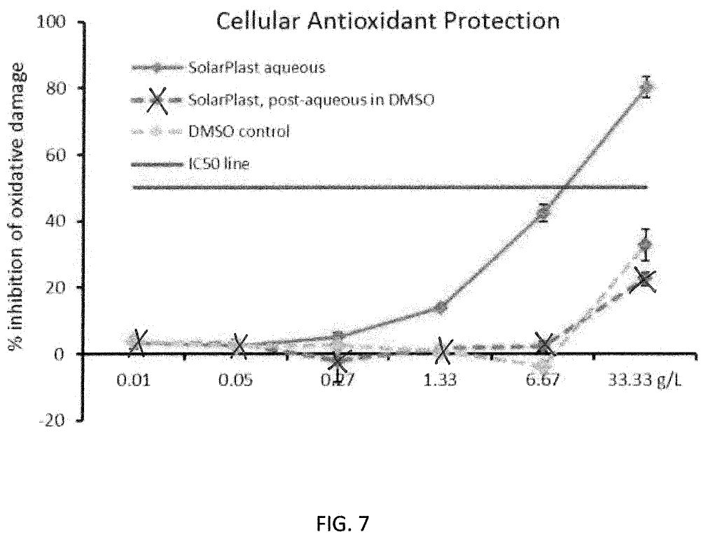

FIG. 7 describes the cellular antioxidant protection by SOLARPLAST® comparing the aqueous fraction to the post-aqueous DMSO extract: Results from the CAP-e bioassay. The percent inhibition of cellular oxidative damage is shown as the average±standard deviation of duplicate data points for each dose of test product. Cell lysing occurred with the highest concentration in each of the post-aqueous DMSO extract and DMSO control. Cell lysing can happen at higher doses of test products that for various reasons are not well tolerated by the live cells. Lysing can be caused by unfavorable pH, salt concentration and other factors.

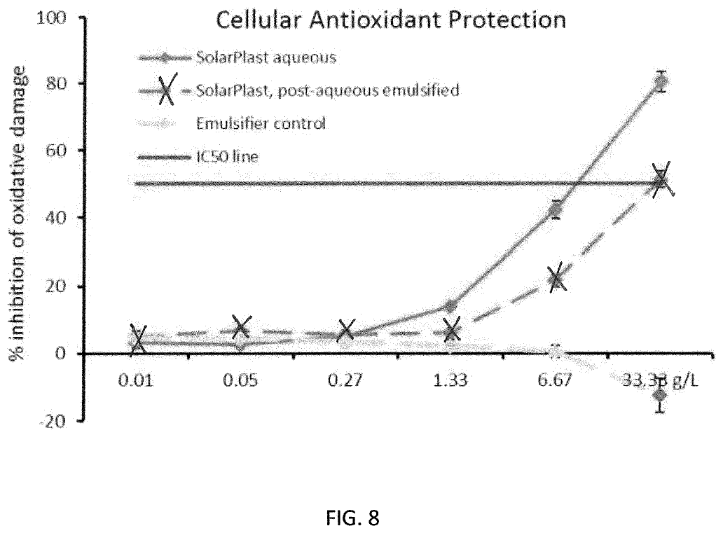

FIG. 8 describes the cellular antioxidant protection by SOLARPLAST® comparing the aqueous fraction to the post-aqueous emulsified extract: Results from the CAP-e bioassay. The percent inhibition of cellular oxidative damage is shown as the average±standard deviation of duplicate data points for each dose of test product. Cell lysing occurred with the highest concentration in the emulsifier control. Cell lysing can happen at higher doses of test products that for various reasons are not well tolerated by the live cells. Lysing can be caused by unfavorable pH, salt concentration and other factors.

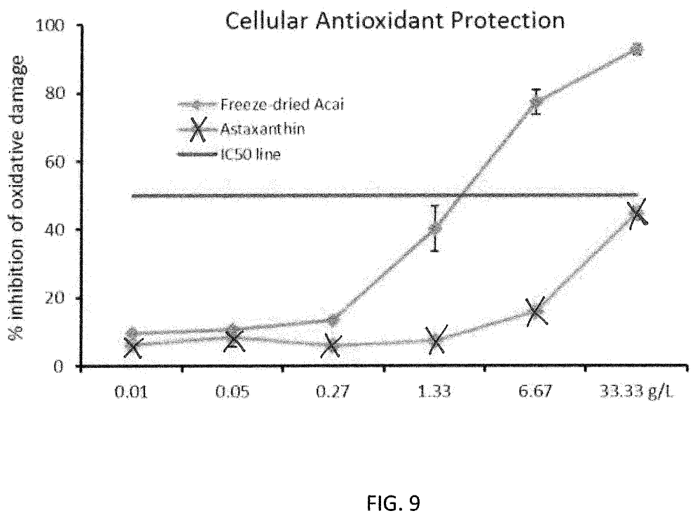

FIG. 9 describes the cellular antioxidant protection by freeze-dried Acai and Astaxanthin: Results from the CAP-e bioassay. The percent inhibition of cellular oxidative damage is shown as the average±standard deviation of duplicate data points for each dose of test product.

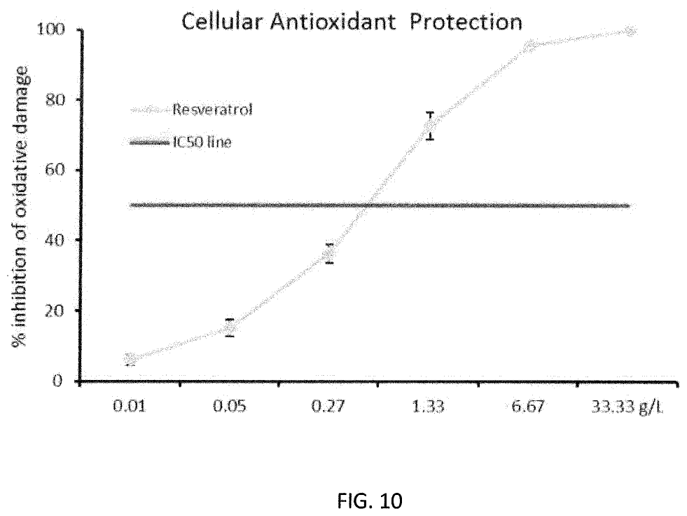

FIG. 10 describes the cellular antioxidant protection by Resveratrol: Results from the CAP-e bioassay. The percent inhibition of cellular oxidative damage is shown as the average±standard deviation of duplicate data points for each dose of test product.

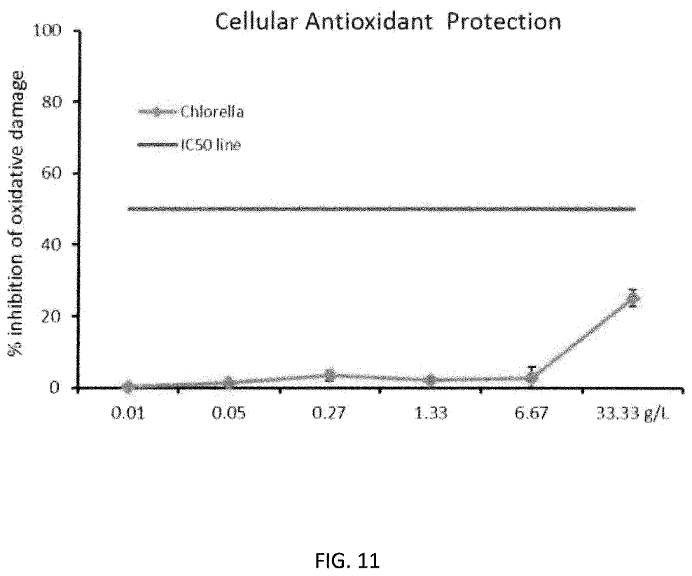

FIG. 11 describes the cellular antioxidant protection by Chlorella: Results from the CAP-e bioassay. The percent inhibition of cellular oxidative damage is shown as the average±standard deviation of duplicate data points for each dose of test product.

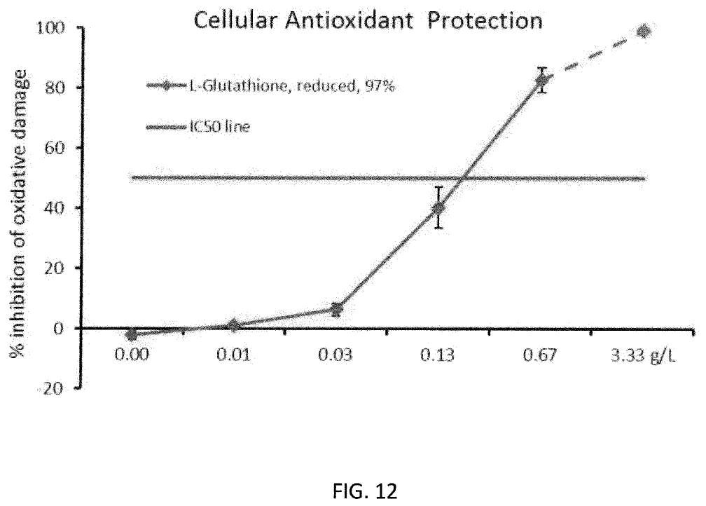

FIG. 12 describes the cellular antioxidant protection by reduced L-Glutathione: Results from the CAP-e bioassay. The percent inhibition of cellular oxidative damage is shown as the average±standard deviation of duplicate data points for each dose of test product. Cell lysing occurred with the highest concentration of reduced L-Glutathione. Cell lysing can happen at higher doses of test products that for various reasons are not well tolerated by the live cells. Lysing can be caused by unfavorable pH, salt concentration and other factors.

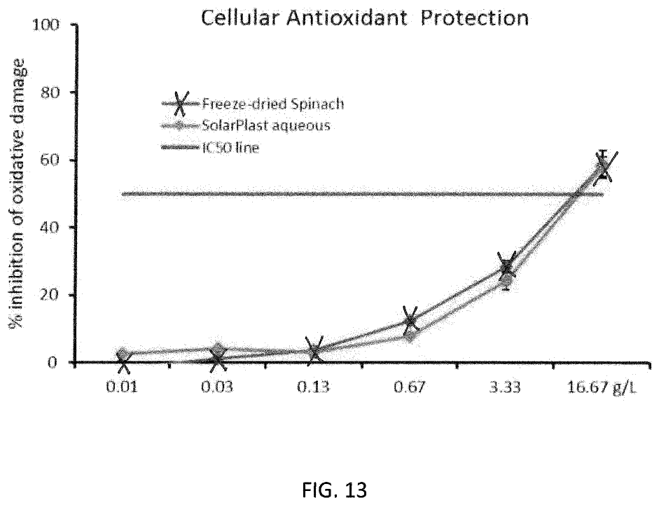

FIG. 13 describes the cellular antioxidant protection by the aqueous fractions of freeze-dried spinach versus SOLARPLAST®: Results from the CAP-e bioassay. The percent inhibition of cellular oxidative damage is shown as the average±standard deviation of duplicate data points for each dose of test product.

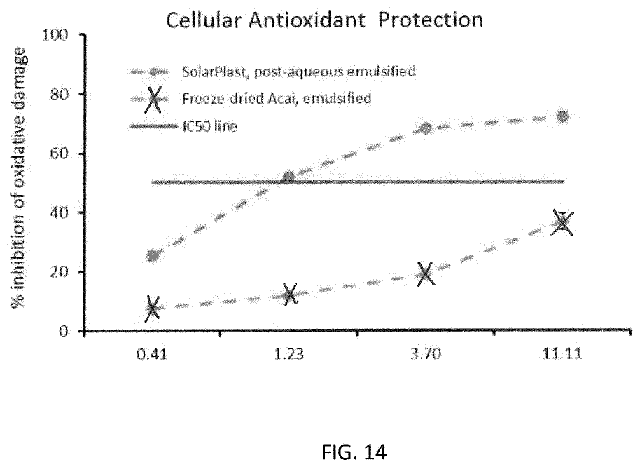

FIG. 14 describes the cellular antioxidant protection by the post-aqueous emulsified extracts from SOLARPLAST® versus Acai: Results from the CAP-e bioassay. The percent inhibition of cellular oxidative damage is shown as the average±standard deviation of duplicate data points for each dose of test product.

In measuring the effect on free radical formation by inflammatory cells, human polymorphonuclear (PMN) cells were used for testing effects of a product on ROS formation. PMN cells constitute approximately 70% of the white blood cells in humans. PMN cells produce high amounts of ROS upon certain inflammatory stimuli.

Natural products may affect PMN cell ROS formation by three different mechanisms: 1. Neutralizing ROS by direct antioxidant affect; 2. Triggering an anti-inflammatory cellular signal, leading to reduced ROS formation; 3. Triggering an immune reaction, leading to enhanced ROS formation.

The effects of test products were measured by exposing freshly purified human PMN cells to the test products. During the incubation with a test product, any antioxidant compounds able to cross the cell membrane can enter the interior of the PMN cells, and compounds that trigger a signaling event can do so. Then the cells are washed, loaded with the DCF-DA dye, which turns fluorescent upon exposure to ROS. Formation of ROS is triggered by addition of H₂O₂. The fluorescence intensity of the PMN cells is evaluated by flow cytometry. The low fluorescence intensity of untreated control cells serves as a baseline and PMN cells treated with H₂O₂ alone serve as a positive control.

If the fluorescence intensity of PMN cells exposed to an extract, and subsequently exposed to H₂O₂, is reduced compared to H₂O₂ alone, this indicates that a test product has anti-inflammatory effects.

In contrast, if the fluorescence intensity of PMN cells exposed to a test product is increased compared to H₂O₂ alone, this indicates that a test product has pro-inflammatory effects by enhancing this aspect of anti-microbial immune defense mechanisms.

The testing was performed on cells from a healthy donor. The low fluorescence intensity of untreated control cells served as a baseline and PMN cells treated with H₂O₂ alone serve as a positive control. If the fluorescence intensity of PMN cells exposed to an extract, and subsequently exposed to H₂O₂, was reduced compared to H₂O₂ alone, this indicated that a test product has anti-inflammatory effects. In contrast, if the fluorescence intensity of PMN cells exposed to a test product was increased compared to H₂O₂ alone, this indicated that a test product had proinflammatory effects by enhancing this aspect of anti-microbial immune defense mechanisms.

Results showed that the SOLARPLAST® aqueous fraction triggered reduction in ROS formation by human inflammatory cells as a measure for anti-inflammatory properties. Both the SOLARPLAST® post-aqueous DMSO-fraction and the DMSO solvent control showed similar anti-inflammatory capabilities, and the properties of the SOLARPLAST® post-aqueous DMSO fraction may be accounted for by the properties of DMSO.

Both the emulsifier control and the post-aqueous emulsified fraction of SOLARPLAST® showed anti-inflammatory properties. This showed that the post-aqueous emulsified fraction of SOLARPLAST® had anti-inflammatory capabilities beyond what was extracted in the aqueous fraction of SOLARPLAST®

Both the aqueous fractions of Freeze-dried Spinach and SOLARPLAST® showed similar antiinflammatory capabilities, with the exception of the highest doses, where Freeze-dried Spinach showed a mild pro-inflammatory effect and the SOLARPLAST® aqueous fraction showed anti-inflammatory effect.

Both Astaxanthin and L-glutathione (reduced) showed less anti-inflammatory capacity than the SOLARPLAST® post-aqueous fraction. L-glutathione (reduced) at higher doses showed robust increase in ROS formation.

Freeze-dried Acai had a similar anti-inflammatory profile to that of the SOLARPLAST® postaqueous emulsified fraction, but had a greater anti-inflammatory capacity compared to the two other fractions of SOLARPLAST®.

Resveratrol and Chlorella showed considerably more anti-inflammatory capabilities than any of the fractions of SOLARPLAST®.

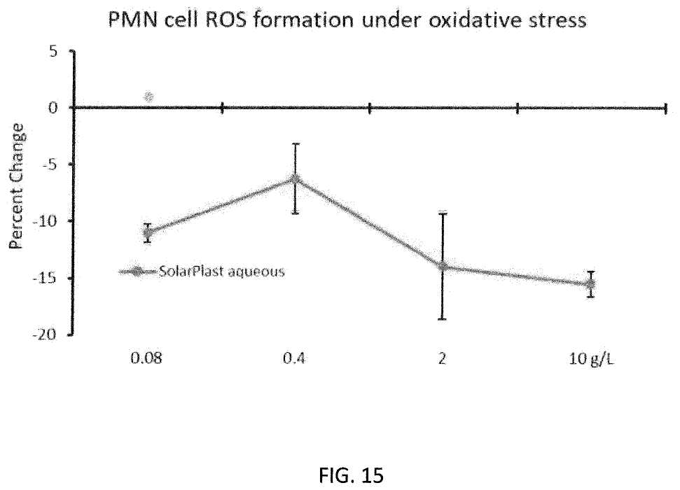

FIG. 15 describes the effects on formation of ROS by human inflammatory PMN cells when exposed to SOLARPLAST®. The data is shown as percent change and as the average±standard deviation of triplicate data points for each dose.

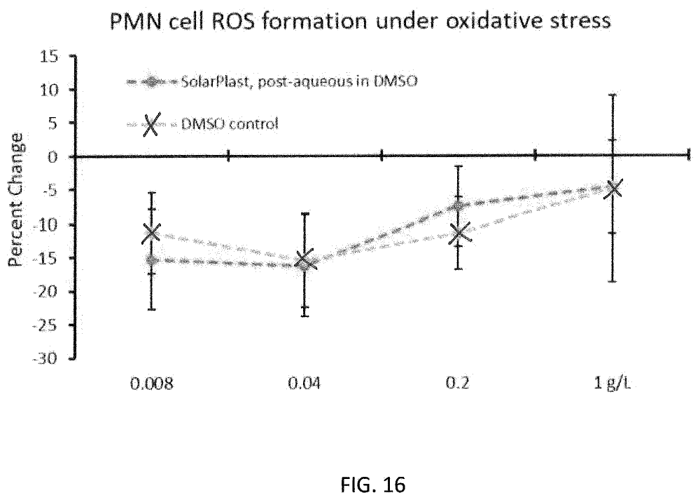

FIG. 16 describes the effects on formation of ROS by human inflammatory PMN cells when exposed to post-aqueous SOLARPLAST® in DMSO. The effects of the solvent DMSO are shown as comparison. The data is shown as the average±standard deviation of triplicate data points for each dose.

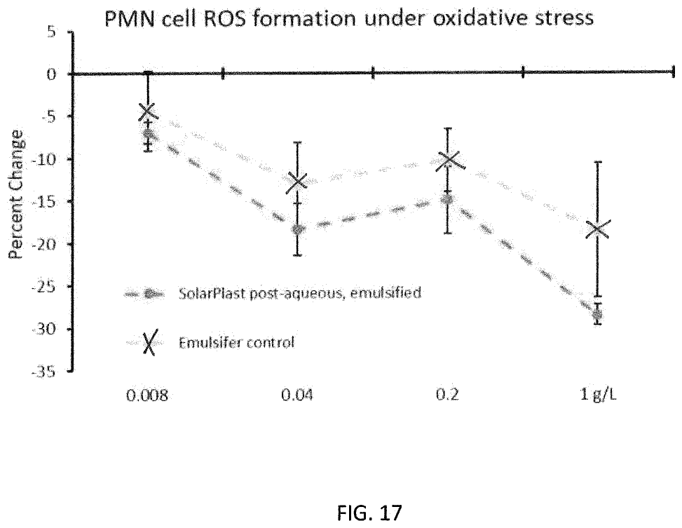

FIG. 17 describes the effects on formation of ROS by human inflammatory PMN cells when exposed to post-aqueous emulsified SOLARPLAST®. The effects of the emulsifier are shown as comparisons. The data is shown as the average±standard deviation of triplicate data points for each dose.

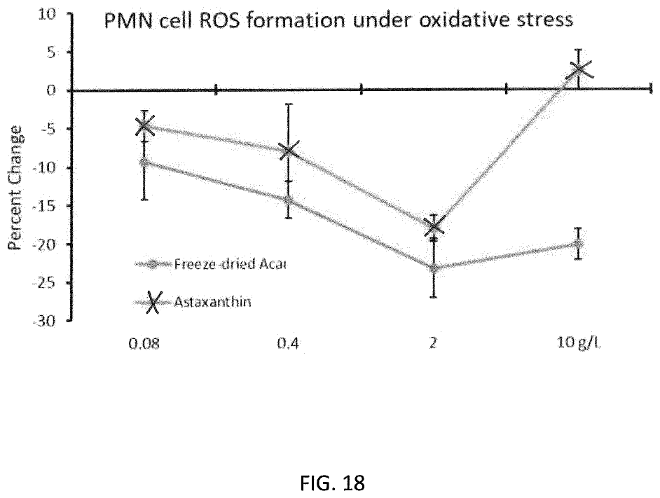

FIG. 18 describes the effects on formation of ROS by human inflammatory PMN cells when exposed to Freeze-dried Acai and Astaxanthin. The data is shown as the average±standard deviation of triplicate data points for each dose.

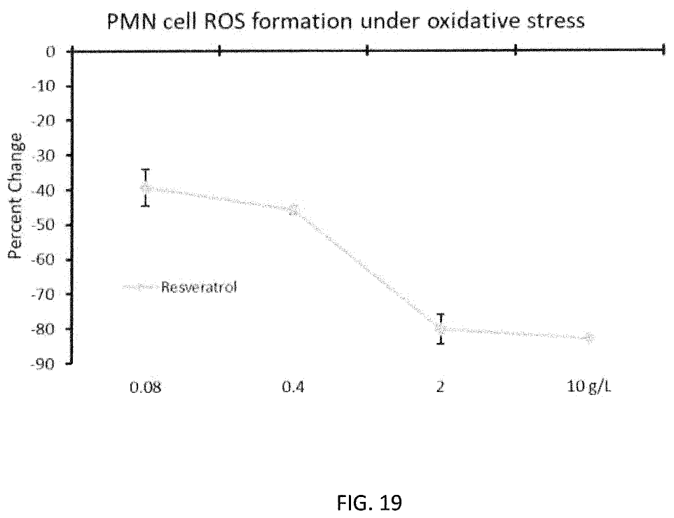

FIG. 19 describes the effects on formation of ROS by human inflammatory PMN cells when exposed to Resveratrol. The data is shown as the average±standard deviation of triplicate data points for each dose.

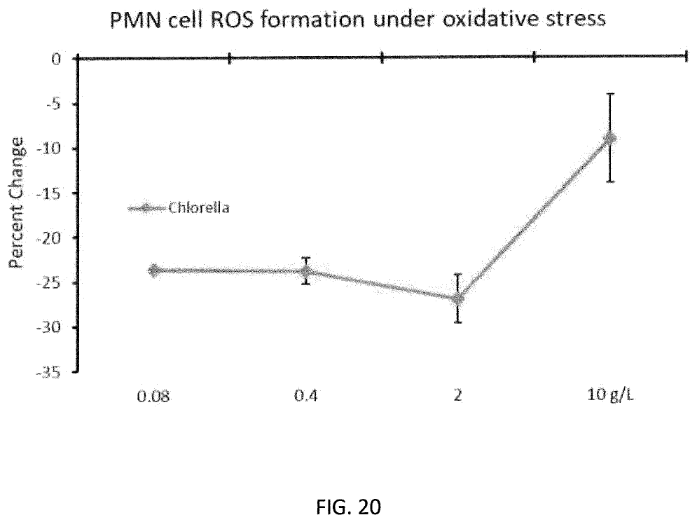

FIG. 20 describes the effects on formation of ROS by human inflammatory PMN cells when exposed to Chlorella. The data is shown as the average±standard deviation of triplicate data points for each dose.

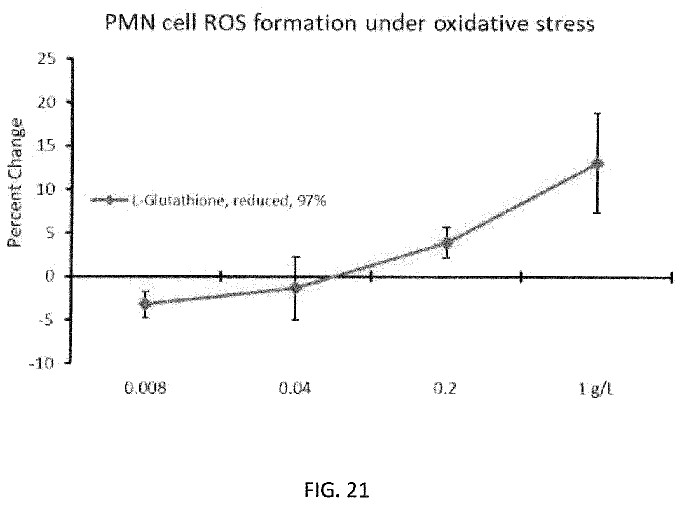

FIG. 21 describes the effects on formation of ROS by human inflammatory PMN cells when exposed to reduced L-Glutathione. The data is shown as the average±standard deviation of triplicate data points for each dose.

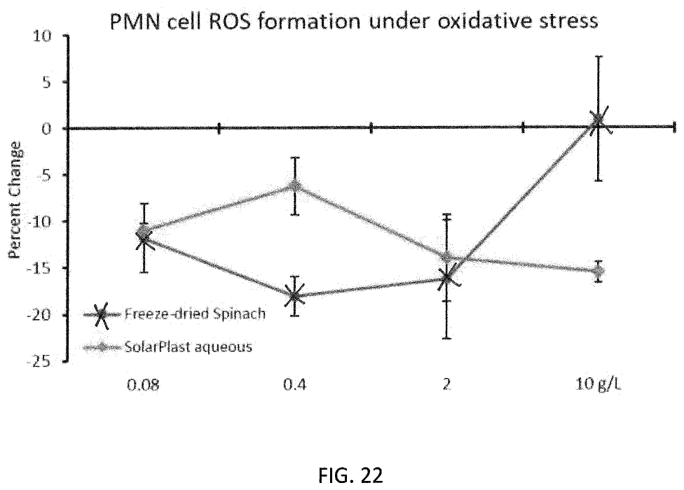

FIG. 22 describes the effects on formation of ROS by human inflammatory PMN cells when exposed to Freeze-dried Spinach and SOLARPLAST®. The data is shown as the average±standard deviation of triplicate data points for each dose.

SOLARPLAST® showed multiple beneficial effects in the cellular bioassays. The aqueous fraction had a good total antioxidant capacity and cellular antioxidant protection and showed a moderate anti-inflammatory effect in the ROS assay. The post-aqueous DMSO fraction did not show interesting results. The post-aqueous emulsified SOLARPLAST® showed interesting properties and performed better than the aqueous fraction in the ROS assay.

When comparing the aqueous fraction of SOLARPLAST® to competing products, SOLARPLAST® had higher total antioxidant capacity than Freeze-dried spinach. SOLARPLAST® provided better cellular antioxidant protection than Astaxanthin and Chlorella. SOLARPLAST® showed stronger inhibition of free radical formation than L-glutathione (reduced), at the dose range tested. Importantly, the biological activity in the post-aqueous emulsified SOLARPLAST® supports the presence of non-aqueous antioxidant and anti-inflammatory compounds, such as compounds from cell membranes.

Antioxidant Assays on SOLARPLAST®

Another study showed various antioxidant assays and measurements of antioxidant enzymes in SOLARPLAST®.

In this study, enzyme activity of various antioxidant enzymes present in spinach were measured. Specifically, the enzymatic activity of superoxide dismutase (SOD), catalase (CAT), peroxidase (POD), glutathione-S-transferase (GST), monodehydroascorbate reductase (MDHAR), and glutathione peroxidase (GPx) were measured. Furthermore, NADP was quantified in SOLARPLAST® and frozen spinach.

Of the materials used in the study, SOLARPLAST® was obtained from Deerland Probiotics & Enzymes, Kennesaw, Ga., USA. Frozen spinach (SuperValu) was purchased from SuperValu, Ireland. Phosphate buffer saline 50× (PBS), SOD assay kit, POD assay kit, Total antioxidant capacity (TOC) assay kit and NADPTotal assay kit were obtained from Sigma-Aldrich, St. Louis, Mo., USA. CAT and MDHAR assay kits were purchased from Cohesion biosciences, London, UK. GST assay kit was bought from Biorbyt, Cambridge, UK. GPx assay kit was obtained from Cayman chemicals, Michigan, USA. Ferric reducing antioxidant power (FRAP) was sourced from Cell Biolabs, San Diego, Californiz, USA. The 2,2-Diphenyl-1-picrylhydrazyl (DPPH) or Trolox equivalent antioxidant capacity (TEAC) assay kit was obtained from Bioquochem, Spain.

Two hundred and fifty milligrams of SOLARPLAST® and frozen spinach were ground to fine powder with liquid nitrogen in a pre-cooled mortar and pestle. The finely ground sample (100 mg) was suspended in 2 ml ice cold PBS (pH 7.4) or relevant assay buffer indicated by manufacturer's instructions. The samples were vortexed vigorously for 1 minute and centrifuged at 9,800×g for 15 minutes at 4° C. Supernatant was removed to a fresh tube and used for each assay.

Total antioxidant activity was assessed from freshly extracted supernatants of SOLARPLAST® and frozen spinach using the TOC, FRAP, and DPPH-TEAC assays. Absorbances were measured at 340 nm, 570 nm, and 520 nm respectively.

The enzymatic activity of SOD was determined from the supernatants of SOLARPLAST® and frozen spinach using the SOD assay kit. All reactions were incubated at 37° C. for 20 minutes before quantifying SOD activity by measuring absorbance decrease at 450 nm.

CAT activity was determined from the supernatants of SOLARPLAST® and frozen spinach resuspended in ice cold assay buffer using CAT assay kit. The reactions were incubated for 3 minutes at room temperature before measuring absorbances at 405 nm.

POD activity was determined from the supernatants of SOLARPLAST® and frozen spinach resuspended in ice cold 1×PBS using the POD assay kit. All reactions were incubated at 37° C. for 120 minutes prior to measuring absorbances at 570 nm.

GST levels were determined from the supernatants of SOLARPLAST® and frozen spinach using the GST assay kit. The concentration of GST was measured by measuring absorbances at 340 nm at an initial time point and after incubating at room temperature for 2 minutes.

MDHAR was determined from the supernatants of SOLARPLAST® and frozen spinach using the MDHAR assay kit. The enzymatic activity was measured at 340 nm after a 2 min incubation at room temperature.

GPx activity was determined from the supernatants SOLARPLAST® and frozen spinach using GPx assay kit. The enzymatic activity was measured once every minute for 10 minutes at a wavelength of 340 nm

NADP_{Total} in SOLARPLAST® and frozen spinach was quantified using NADP/NADPH quantification kit. All reactions were incubated for 240 minutes before reading absorbances at 450 nm.

For TOC in SOLARPLAST® and frozen spinach, the presence of small antioxidant molecules and antioxidant enzymes in SOLARPLAST® and organic frozen spinach was assessed via performing a set of three TOC assays.

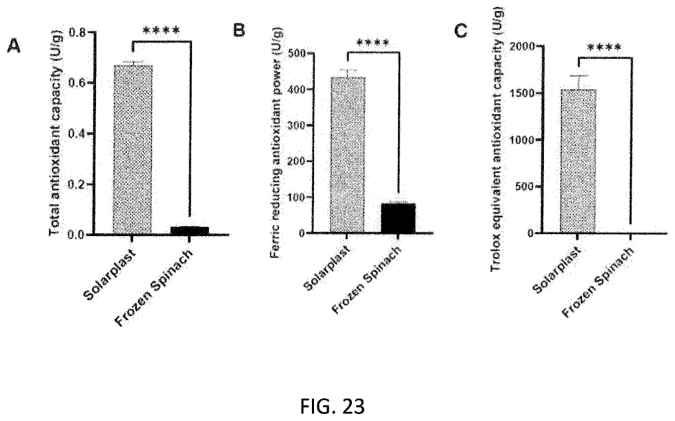

FIG. 23 depicts several representative antioxidant assays, as follows.

FIG. 23A shows through a TOC assay, that SOLARPLAST® had a capacity of 0.66±0.0 U/g compared to 0.03±0.0 U/g in frozen spinach (p<0.0001).

FIG. 23B shows through a FRAP assay, that SOLARPLAST® had a capacity of 433.1±20.7 U/g compared to 83.7±2.9 U/g in frozen spinach (p<0.0001).

FIG. 23C shows through DPPH-TEAC assays, that SOLARPLAST® had a capacity of 1538±143.9 U/g compared to 6.7±0.0 U/g in frozen spinach.

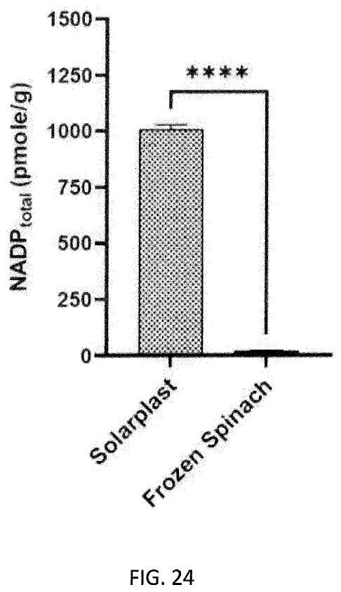

FIG. 24 shows through a NADP/NADPH quantification kit, that SOLARPLAST® had significantly higher concentrations of NADP_{Total} (1007±21.2 pmol/g) than in frozen spinach (18.84±1.6 pmol/g).

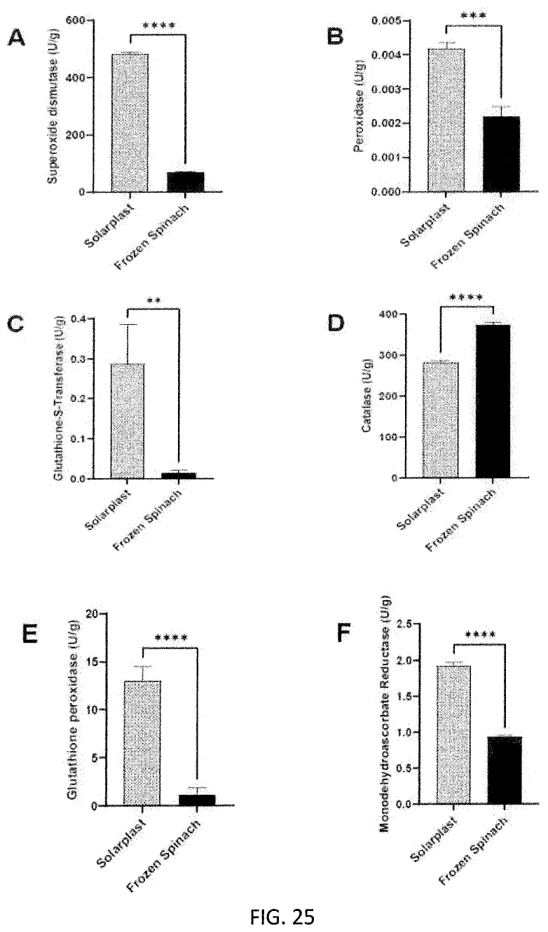

FIG. 25 depicts enzyme activity of various antioxidant enzymes present in spinach comparing SOLARPLAST® with Frozen spinach:

FIG. 25A shows that the level of SOD activity was found to be 483±7.3 U/g in SOLARPLAST® compared to 69±0.1 U/g in frozen spinach (p<0.0001).

FIG. 25B shows that the level of POD activity was found to be 0.004±0.0 U/g in SOLARPLAST® compared to 0.002±0.0 U/g in frozen spinach (p<0.0001).

FIG. 25C shows that the level of GST activity was found to be 0.28±0.1 U/g in SOLARPLAST® compared to 0.01±0.0 U/g in frozen spinach (p<0.0001).

FIG. 25D shows that the level of CAT activity was found to be 282.5±4.5 U/g in SOLARPLAST® compared to 374±6.0 U/g in frozen spinach (p<0.0001).

FIG. 25E shows that the level of GPx activity was found to be 13.1±1.4 U/g in SOLARPLAST® compared to 1.2±0.7 U/g in frozen spinach (p<0.0001).

FIG. 25F shows that the level of MDHAR activity was found to be 1.9±0.0 U/g in SOLARPLAST® compared to 0.9±0.0 U/g in frozen spinach (p<0.0001).

Oxygen Radical Antioxidant Capacity (ORAC)

ORAC is a well-known standard assay. SOLARPLAST® has been tested for Antioxidant capacity against all radicals, and has been shown to have a higher total capacity than plant extracts (raw or frozen).

SOLARPLAST® has also been shown to have a higher total capacity than other very well-known and competitive antioxidant products that have not been enzymatically treated and optimized.

Several enzyme systems within the body neutralize excess free radicals. One important component of the defense system that prevents the body from free radical damage includes antioxidants that can serve as chemical scavengers or quenchers of free radicals. Antioxidants are molecules which can safely interact with free radicals and terminate the chain reaction before vital molecules, or reactive sites, are damaged or chemically modified. Antioxidants are of two types: enzymatic antioxidants and non-enzymatic antioxidants. Enzymatic antioxidants include superoxide dismutase (SOD), glutathione peroxidase (GPx) and catalase (CAT). Non-enzymatic antioxidants include specific bioactive metabolites and several broad classes of agents, such as: ascorbic acid (Vitamin C), α-tocopherol (Vitamin E), glutathione (GSH), carotenoids, flavonoids, polyphenols, and molecules from other natural sources. Under normal physiological conditions, there is a balance between both the activities and the intracellular levels of these antioxidants within a living subject or organism. However, during stress, there may be an imbalance between the required beneficial, protective levels versus the actual physiological levels of these antioxidants in the subject. In these situations, antioxidants from one or more exogenous sources may be needed to overcome the effects of an assault by one or more types of free radicals. An ideal antioxidant should scavenge/neutralize different types of free radicals, including ROS, e.g. OH^{•}, O₂^{•-}, OOH^{•}, NO^{•} and ONOO⁻ (or mixtures thereof), present in the biological systems.

Therefore, in embodiments of the present invention, a battery of in vitro antioxidant screens may be used to assess the comparative antioxidant activities of protoplast containing compositions prepared according to the principles of the present invention. Useful assays may include: ABTS radical cation decolorisation assay (N. Pellegrini, et al., J. Nutr. (2003) 133: 2812-2819); DPPH radical decolorisation assay (I. Gulcin, et al., J. Ethnopharmacol. (2004) 90: 205-215); FRAP—Ferric reducing assay for plasma: Assay for reduction power (N. Pellegrini, et al.); Hydroxyl radical scavenging assay by 2-deoxyribose degradation method (S. K. Chung, et al., Biosci. Biotech. Biochem. (1997) 61: 118-123); Hydrogen peroxide scavenging assay (I. Gulcin, et al.); Estimation of total phenolic content by Folin-Ciocalteu method (S. A. L. Morais, et al., J. Braz. Chem. Soc. (1999) 10:447-452); Nitric oxide radical scavenging assay (R. Sundararajan, et al., Complementary & Alternative Medicine (2006) 6: 8-14); Peroxynitrite scavenging assay (L. B. Valdez, et al., Medicina (Lithuania), (2007) 43: 306-309); Inhibition of rat erythrocyte membrane lipid peroxidation (T. Moriguchi, et al., J. Nutr. (2001) 131: 1016S-1019S); and, Inhibition of lipid peroxidation of goat brain homogenate (S. K. Mondal, et al., Ind. J. Exp. Biol. (2006) 44: 39-44). Superoxide radical scavenging assay is assessed by NBT (Nitro Blue TetraZolium)-hypoxanthine/XO method (Palanisamy, et al., “Rind of the rambutan, Nephelium lappaceum, a potential source of natural antioxidants.” Food Chemistry (2008) 109:54-63). Yet another superoxide radical scavenging assay is assessed by NBT (Nitro Blue TetraZolium)-NADH (Nicotinamide Adenine Dinucleotide-Reduced)-PMS (Phenazonium Methosulphate) method (I. Gulcin, et al., J. Ethnopharmacol. (2004) 90: 205-215, as above).

As described herein, the protoplast enriched composition (SOLARPLAST®) captures the energy of the sun and delivers it to you in 1 simple to swallow capsule. SOLARPLAST® provides energy derived directly from the sun along with a powerful natural antioxidant function that repairs damage from generating and using energy during workouts, for example. Light to energy converting organelles encased in endogenous lipid bilayers provide vital nutrient components that are protected in their own biological coating, i.e. within the protoplasts. The mechanistic roles of these organelles cause natural concentrations of antioxidant components including glutathione reductase, NADPH and FAD. SOLARPLAST® contains photosynthetic complexes containing naturally high concentrations of ATP, NADPH, ADP, AMP, NADP, Niacin, B12, Adenine, Ribose, etc. These energy molecules can be absorbed directly or processed, then absorbed and quickly reformed to energy molecules that a human body can use for physical activity. Once ATP enters the body, the ATP is broken down into two other compounds: adenosine and inorganic phosphate. These compounds are incorporated into and expand the body's ATP pools. The ATP pools supply adenosine to the red blood cells, which use it as a building block to manufacture ATP. Then, the expanded red blood cell ATP pools are slowly released into the blood plasma. A variety of published pre-clinical and human clinical trials have found that extracellular ATP and its major degradation product, adenosine, activate specific ATP and adenosine receptors on the cells that line the blood vessel walls, improving: (1) blood vessel tone and increasing vasodilation; (2) the flow of blood to the heart, brain and peripheral areas, especially to skeletal muscles; (3) the delivery of glucose, nutrients and oxygen to working and recovering muscles; and (4) removal of catabolic waste products.

Each protoplast in the present composition may contain multiple chloroplasts. Chloroplasts serve as the energy factory of the food chain, providing high levels of many energy components the body can use to make energy quickly and efficiently when you need it most, e.g. niacin, vitamin B12, NADP⁺, etc.

The recommended daily dose for SOLARPLAST® is between 100-150 milligrams for a healthy adult.

The protoplasts of this invention can be derived from spinach species. Other useful plants or plant sources include kale, hemp, collard greens, mustard greens, arugula, swiss chard, bok choy leaves, turnip greens and any other green leafy plant, and the like.

A biochemical method is provided to release the protoplasts (altered forms of plant cells from which the cell wall has been partially or completely removed) to provide components that are needed to promote health. Certain plant cells are enzymatically treated to release cell contents and ultimately freeze dried to produce a powder. The dried protoplast enriched mixture or a composition thereof may be used for its antioxidant properties contained therein.

Preparation of Protoplasts

This procedure is generally outlined in the summary section above.

In its principal embodiment, protoplasts are prepared using an enzymatic method to obtain inner cell organelles which can provide antioxidant and antiaging activity for dietary supplements.

Using a blend of cell wall specific enzymes (0.1-50% by wt based on total plant weight) (such as cellulase, hemicellulose, and pectinase), osmotic protectants (0.01-50% by wt based on total plant weight) (such as sugars and derivatives, amino acids and derivatives and polyols and derivatives) and other bulk density enhancing non-active ingredients such as carbohydrates, fibers etc (0.01-50% by wt based on total plant weight), a reaction is carried out in kettles at a temperature range of 30-37° C. Plants can be added such as kale, spinach, hemp, collard greens, mustard greens, arugula, swiss chard, bok choy leaves, turnip greens and any other colorful plant. Protoplasts are the organelles left once the cell wall is disintegrated. Antioxidant and chaperone activity of the remaining part of the plant is tested using assays validated to test antioxidant activity in foods, e.g. ORAC assay.

The method will be performed as follows: Water will be added and mixed on highest setting and turn the heat on. Osmotic protectant is added to kettle and heat. Plants are measured out at 0.1-50% by wt and added to kettle. While the plant is weighed, cell wall degrading enzymes will be added to water at 0.1-50% by wt based on plant weight until fully dissolved. There should be no visible chunks of material once it is mixed. After the kettle filled with plant material reaches 30° C.-37° C., the enzyme mixture is added and the timer is started for 0-2 hours. After heating the temperature should be between 10° C.-15° C. and then bulk density enhancer is added. Rolling pins are used to roll and crush the product in a sealed Mylar bag taking care to crush both sides evenly and until contents feel mostly broken up and no larger pieces remain.

Useful additional parameters are as follows.

The addition of the enzyme starts the clock, and at 25 mins bulk density enhancing material is added.

The bulk density enhancing material is added and the temperature is turned off. The mixture is mixed approx. another 5 mins to ensure it is mixed well.

The product may be loaded and freeze-, drum-, spray- or refractive dried using standard techniques.

Useful enzymes include cellulase, pectinase and xylanase.

Useful osmotic protectants include erythritol, trehalose, sorbitol, or the like, or any other similar sugar alcohol.

Useful bulk density enhancers include organic fibers and oligosaccharides. Useful bulk density enhancing materials may include, but are not limited to, fructo-oligosaccharides (FOS), pectins, carrageenan, starches, dextrose, or polydextrose.

### Industrial Example

For each dryer load, the temperature is monitored to make sure it never exceeds 37° C.

Step 1. Water is added to a reactor kettle for about 4.5 minutes using a hose.

Step 2. Osmotic protectant is measured out and mixed in bucket of hot water until dissolved.

Step 3. Osmotic protectant solution is added to the reactor kettle under heating.

Step 4. Spinach plant material weighed out and added to the reactor kettle.

Step 5. Cell wall degrading enzyme is mixed with water and the resulting mixture is add to the reactor kettle.

Step 6. Once the established time of 25 minutes has passed bulk density enhancer is added. A combination of mixing means e.g. a paddle may be used to get the reaction products mixed well.

Step 7. Once bulk density enhancer is mixed in, the process it initiated to tray up for drying.

Using the aforementioned method for obtaining protoplasts is a cost-efficient way of obtaining the active ingredients inside the cell wall thus providing the concentrated active ingredients in smaller quantities. The present innovative way of using cell wall degrading enzymes with osmotic protectants allow the intact organelles to provide benefit in a supplement form. Currently cell wall degrading methods are only used to insert DNA, not to obtain intact organelles for the use of its antioxidant and anti-inflammatory properties.

Depending on the weight percentage of spinach or other plant material used, the percentage of cell degrading enzymes and bulk density enhancing material is determined. Further, this process needs to be able to be done at the respective temperatures with the right amount of water, in large quantities taking care not to overheat and brown the plant, thus obtaining useless bioactive organelles. That is, the operator must exercise due care not to reduce or compromise biological activity in the final product composition.

In certain embodiments, a nutraceutical comprising SOLARPLAST® alone or in combination with vitamins may be any variety of food or drink. For example, nutraceuticals may include drinks such as nutritional drinks, diet drinks as well as sports, herbal, and other fortified beverages. Additionally, nutraceuticals may include foods intended for human or animal consumption such as baked goods, for example, bread, wafers, cookies, crackers, pretzels, pizza, and rolls, ready-to-eat breakfast cereals, hot cereals, pasta products, snacks such as fruit snacks, salty snacks, grain snacks, nutrition bars, and microwave popcorn, dairy products such as yogurt, cheese, and ice cream, sweet goods such as hard candy, soft candy, and chocolate, beverages, animal feed, pet foods such as dog food and cat food, aqua-culture foods such as fish food and shrimp feed, and special purpose foods such as baby food, infant formulas, hospital food, medical food, sports food, performance food or nutritional bars, or fortified foods, food preblends or mixes for home or food service use, such as preblends for soups or gravy, dessert mixes, dinner mixes, baking mixes such as bread mixes, and cake mixes, and baking flower. In certain embodiments, the food or beverage does not include one or more of grapes, mulberries, blueberries, raspberries, peanuts, milk, yeast, or extracts thereof.

In certain embodiments, SOLARPLAST® alone or in combination with vitamins of the present invention to a mammal (e.g., human) in need thereof, and methods of treating and/or preventing symptoms, diseases, disorders, or conditions associated with, or having etiologies involving, inflammation and/or that would benefit from increased mitochondrial activity in a mammal (e.g., human) comprise delivering or administering compositions described herein.

Oral formulations of SOLARPLAST® are contemplated. Useful therapeutic dosages of can be used in a human individual, daily, given in one or more doses. Another suitable dose range is from about 100 mg to about 1000 mg. Another suitable dose range is from about 5 mg to about 500 mg. Another suitable dose range is from about 50 mg to about 500 mg.

Compositions for oral formulations useful for delivering a dietary supplement composition comprising SOLARPLAST® alone or in combination with vitamins that are palatable to mammals (e.g., humans) are known in the art. The compositions can be orally administered, for example, with an inert diluents or with an compatible edible carrier, or it can be enclosed in hard or soft shell gelatin capsules, or it can be compressed into tablets, or it can be incorporated directly with the food of the diet. For oral administration, the dietary composition may be incorporated with an excipient and used in the form of ingestible tablets, buccal tablets, troches, capsules, elixirs, suspensions, syrups, wafers, and the like. The tablets, troches, pills, capsules, and the like can also contain the following: a binder such as gum tragacanth, acacia, corn starch, or gelatin; excipients such as dicalcium phosphate; a disintegrating agent such as corn starch, potato starch, alginic acid, and the like; a lubricant such as magnesium stearate; and a sweetening agent such as sucrose, lactose, or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the active compound, sucrose as a sweetening agent, methyl and propylparabens as preservatives, a dye, and flavoring such as cherry or orange flavor. Oil-in-water emulsions may be better suited for oral use in infants or children because these are water-miscible, and thus their oiliness is masked. Such emulsions are well known in the pharmaceutical and nutritional sciences.

Clinical Study

A placebo-controlled human clinical trial was conducted (NCT04144777) in 68 healthy individuals and 16 smokers in which the daily dose of SOLARPLAST® was 100 mg/day over 45 days.

Spinach, or Spinacia oleracea, contains numerous active components, including flavonoids, which exhibit antioxidative and anti-inflammatory properties. SOLARPLAST® is an enzymatically treated spinach extract, which is derived from Spinacia oleracea. This study was designed to investigate the effects of 45 day SOLARPLAST® supplementation on metabolic safety, oxidative stress, and inflammation, while secondary assessments included questionnaires associated with skin, physical, and mental health in smokers and nonsmokers. A total of 68 healthy, non-smoking individuals (25.7±9.7 yrs) and 16 smokers (28.1±13.9 yrs) completed the study. Participants were divided into non-smokers and smokers and randomly assigned to consume either once daily SOLARPLAST® (100 mg/day; 1×106 light converting units [LCUs]) or placebo for 45 days. Significant decreases were found for TNF-α, perceived pain, skin problems, anxiety and irritability (p<0.05) in SOLARPLAST® supplemented non-smoking individuals. Significant reductions were found for ROS/RNS, IL-6, and depression in SOLARPLAST® supplemented smokers (p<0.05). Forty-five days of daily SOLARPLAST® supplementation positively impacts inflammation and perceived mood-related outcomes in both smokers and non-smokers, while also specifically impacting oxidative stress in smokers.

SOLARPLAST® is a recently developed nutritional supplement derived from Spinacia oleracea and its efficacy has been tested in humans herein. Moreover, SOLARPLAST® is an enzymatically treated spinach extract produced by way of raw frozen spinach undergoing processing with specific conditions in the presence of an enzyme formulation. The process is ended by freeze-drying the resultant spinach digest to produce a lyophilized spinach extract, as shown above. The mechanistic components of SOLARPLAST® consist of natural concentrations of antioxidants normally found in the body including glutathione reductase, NADPH, and FAD. Additionally, SOLARPLAST® contains photosynthetic complexes with high concentrations of ATP, NADPH, ADP, AMP, NADP, niacin, B12, adenine, and ribose. The unique mixture of molecules is thought to attack oxidants through chaperone activity.

The present study was therefore undertaken to evaluate the possible effects of chronic daily consumption of SOLARPLAST® for 45 days by healthy, non-smoking individuals and a small cohort of smokers. Smokers were selected as a subgroup as it is well known that the use of cigarettes enhances oxidative stress, not only by mounting reactive oxygen species but also through weakening antioxidant defense systems. Primary outcomes of this investigation included a safety profile (i.e., metabolic panel), and oxidative stress and inflammatory-related markers, while secondary assessments included questionnaires associated with skin, physical, and mental health.

Materials and Methods

The study was prospectively registered as a Clinical Trial (NCT04144777) and was approved by the university's institutional review board (IRB #19-165), Kennesaw State University, Kennesaw, Ga. Participation consisted of two visits to the laboratory, separated by a 45-day supplementation period (SOLARPLAST® or placebo). During the supplementation period, participants completed two phone call check-ins with a member of the research team, as well as logged their physical activity and dietary intake throughout their enrollment. The following primary outcome variables were specified a priori: blood biomarkers (complete metabolic panel, inflammation [TNF-α, IL-4, IL-6], oxidative stress [glutathione, reactive oxygen species to reactive nitrogen species (ROS/RNS)] and perception of skin, physical, mental, and lifestyle outcomes via questionnaires. All data was collected in accordance with the Declaration of Helsinki.

Study Design

Participants were recruited if they were 1) between the ages of 18-55 years, 2) smokers or non-smokers, 3) currently weight stable, 4) not following a specific type of diet (e.g., ketogenic, vegetarian, fasting, etc.), 5) not consuming antibiotics or anti-inflammatory medications, 6) not an athlete (competing on collegiate or professional sports teams), 7) no self-report of chronic disease or illness, including heart disease, chronic lung disease, diabetes, kidney disease or related, and 8) if a smoker—not attempting to quit or using smoking cessation products. Exercise was also taken into consideration with non-smokers recruited if they self-reported exercising at least 2 days per week, but no more than 4 days and smokers not exercising at all. Smoking was defined as individuals who had smoked every day for the last year at the time of enrollment—termed “everyday smokers”. Further, participants were excluded if they missed 3 consecutive or 5 or more total days of the supplement, if they became pregnant, if they stopped smoking (if originally enrolled in the smoking group), and if they started to take any new medications.

A total of 68 non-smoking individuals (28 males and 40 females) and 16 smokers (6 males and 10 females) completed the study of the 74 non-smokers and 19 smokers who were enrolled. Participants were divided into non-smokers and smokers and randomly assigned to consume either once daily SOLARPLAST® (100 mg/day; 1×106 light converting units [LCUs]) or placebo (100 mg/day maltodextrin). Supplements were identical in texture, size, color, shape, and taste. Participant demographics may be found in Table 3.

TABLE 3

| | Solarplast ® | Placebo | Solarplast ® | Placebo |
|---|---|---|---|---|
| | Non-Smoker | Non-Smoker | Smoker | Smoker |
| Age (yrs) | 25.7 ± 9.7 | 27.2 ± 13.4 | 28.1 ± 13.9 | 31.6 ± 13.4 |
| Height | 168.0 ± 9.3 | 165.5 ± 8.3 | 167.7 ± 16.7 | 167.3 ± 9.5 |
| (cm) | | | | |
| Body | 71.7 ± 17.4 | 67.6 ± 13.2 | 73.4 ± 18.6 | 76.4 ± 16.0 |
| Mass (kg) | | | | |
| Body | 27.4 ± 4.6 | 26.1 ± 5.4 | 26.2 ± 6.5 | 29.3 ± 6.5 |
| Fat (%) | | | | |
| Exercise | 127.7 ± 30.2 | 135.7 ± 32.5 | 0.0 ± 0.0 | 0.0 ± 0.0 |
| (min/ | | | | |
| week) | | | | |

Smoker and non-smoker participant group characteristics (mean ± SD).
SOLARPLAST ® non smoker (females n = 22; males n = 12); Placebo non-smoker (females n = 16; males n = 18); SOLARPLAST ® smoker (females n = 5; males n = 3); Placebo smoker (females n = 5; males n = 3).

Participants arrived at the laboratory in a fasted state (8-12 hours) for both visit 1 (Pre) and visit 2 (Post). Sessions took place in the morning, between the hours of 8:00 AM and 12:00 PST. All visits for a given participant fell at approximately the same time (+/−2 hours). Upon arrival for Pre, participants were informed of the study procedures, risks and benefits, and gave their verbal and written consent. They then filled out a health history questionnaire consisting of medical, physical activity, diet, and supplementation history, as well as a 24-hour food recall. Immediately following, participants had their anthropometrics and body composition assessed followed by a blood draw. The visit was ended with the completion of questionnaires: skin visual analog scale (SKN-VAS) and Anti-Aging QOL Common Questionnaire (AAQOL). Prior to leaving, participants were given their random assignment supplement and instructions on how to take it each day for 45 days, dates of follow-up visits (two phone call check-ins and Post-visit), and were able to ask questions. Participants then returned to the laboratory 45 days later for Post testing, which consisted of supplement compliance, anthropometrics, and body composition, blood draw, and questionnaires.

Phone call visits were completed by a member of the research team on days 15 and 30 of supplementation. Visits were intended to check on supplementation compliance (i.e., missed days taking the supplement) and check for adverse symptoms: 1) skin: flushing/redness/warmth; itching; rash (appearance and location); throat symptoms; cough; back pain; abdominal pain; nausea/vomiting/diarrhea; fever; joint (pain/swelling/redness/stiffness); numbness/tingling; changes in blood pressure; dizziness; tunnel vision; loss of consciousness; confusion; other (please specify).

Body mass and height was assessed via a standard scale (WB-3000 Digital Scale, Tanita, Tokyo, Japan) without shoes. Body fat percentage was collected via bioelectrical impedance analysis (BIA; InBody770, InBody Co., Seoul, Korea) for all participants. Each individual was asked to wipe their hands and feet prior to the InBody 770 assessment (InBody Tissue) to enhance electrical conductivity. All assessments occurred in the fasted state (8-12 hours).

Single venipunctures at the antecubital space were used to collect blood samples from participants. Approximately 20-25 mL of blood was taken from participants at each collection time. Samples were collected into Vacutainer™ tubes containing ethylenediaminetetraacetic acid (EDTA) (inverted 8-10 times prior to processing) and serum separator (SST). SST tubes were set aside to clot for 10 minutes prior to centrifugation. Two separate centrifugation processes occurred. A single SST tube was centrifuged at room temperature (LabCorp Centrifuge Horizon Model 642E, Drucker Diagnostics, Port Matilda, Pa.) and picked up by a LabCorp representative for comprehensive metabolic panel assessment. The metabolic panel consisted of the assessment of glucose, blood urea nitrogen (BUN), creatinine, estimated glomerular filtration rate (EGFR), blood urea nitrogen-creatinine ratio, sodium, potassium, chloride, total carbon dioxide, calcium, total protein, albumin (A), total globulin (G), A/G ratio, total bilirubin, alkaline phosphatase, aspartate aminotransferase (AST), and alanine aminotransferase (ALT). The remaining SST and EDTA tubes were centrifuged at 1650×g for 10 minutes at 4° C. The resulting serum and plasma was aliquoted and stored in −80° C. freezer until analysis. Samples were aliquoted into multiple vials as to not repeat freeze-thaw cycles. Samples were used to analyze markers of oxidative stress and inflammation per manufacturer guidelines and using a Varioskan LUX Multimode Microplate Reader (Thermofisher, Waltham, Mass., USA): ROS/RNS (OxiSelect in Vitro ROS/RNS Assay Kit, STA-347-5, plasma), glutathione (Cayman Chemical, 703002, plasma), IL-6 (Invitrogen, BMS213HS; serum), IL-4 (Invitrogen, KHC0041; serum), and TNF-α (Invitrogen, BMS223HS; serum).

The SKN-VAS was used to assess participant's perception of skin changes and consisted of the following on a 5-inch scale: 1) dryness of skin, 2) flushing of the skin, 3) inconsistency of skin with make-up (if applicable), 4) itching of the skin, 5) eczema, 6) wrinkles: body, 7) wrinkles: face, 8) coarse skin, 9) softness of skin, 10) elasticity of the skin, 11) glossy skin, 12) overall complexion of the skin. Participants were asked to mark vertically on the SKN-VAS line for each question. The Anti-Aging Common Questionnaire (AAQOL) was also collected and consisted of 32 question items of physical symptoms, 21 questions concerning mental symptoms, and lifestyle-related questions, including sleep, drinking, smoking, and exercise habits. Both physical and mental symptoms were collected as a 5-point Likert scale.

Statistical Analysis

Data are reported as mean±standard deviation. Baseline group characteristics were compared using independent t-tests. Separate analyses were run for each group, non-smokers and smokers. Data were analyzed via a 2 (SOLARPLAST® vs Placebo)×2 (Pre and Post) analysis of variance (ANOVA). If significant group x time interactions were detected, post-hoc paired sample t-tests were utilized to identify where differences occurred with a Bonferroni adjustment. Significance was set a priori at an alpha level of p<0.05. Statistical analyses were performed using SPSS Version 27.

Results

Physical characteristics did not differ between SOLARPLAST® and placebo groups (p>0.05) (Table 3). No participants reported a problem associated with the ingestion of either supplement (i.e., placebo or SOLARPLAST®), and all conditions appeared to be well-tolerated.

A comprehensive metabolic panel was determined for each group, non-smokers and smokers. For non-smokers, there were no significant changes for glucose, BUN, BUN to creatinine ratio, sodium, potassium, chloride, carbon dioxide, calcium, total protein, albumin, globulin, A/G ratio, bilirubin, AST, and EGFR. There was a significant group x time interaction for creatinine (p=0.016) and alkaline phosphatase (p<0.01); however, post-hoc evaluation indicated no significant differences (p>0.05). For smokers, there were no significant changes for glucose, BUN, creatinine, BUN to creatinine ratio, sodium, potassium, chloride, carbon dioxide, calcium, total protein, albumin, globulin, A/G ratio, bilirubin, alkaline phosphatase, AST, ALT, and EGFR.

Metabolic panel results are shown in Table 4. Comprehensive metabolic data at Pre and Post are included from non-smokers and smokers who consumed SOLARPLAST® or Placebo for 45-days. SOLARPLAST® non-smoker (females n=22; males n=12); Placebo non-smoker (females n=16; males n=18); SOLARPLAST® smoker (females n=5; males n=3); Placebo smoker (females n=5; males n=3).

TABLE 4

| | Solarplast ® | | Placebo | |
|---|---|---|---|---|
| | Non-Smoker | | Non-Smoker | |
| | Pre | Post | Pre | Post |
| Glucose (mg/dL) | 90.34 ± 7.5 [symbol] | 90.76 ± 7.22 | [symbol] 7.94 ± 7.16 | 88.80 ± 6.50 |
| BUN (mg/dL) | 12.09 ± 3.05 | 12.36 ± 3.89 | 12.60 ± 3.98 | 11.97 ± 3.65 |
| Creatinine (mg/dL) | 0.87 ± 0.19 | 0.91 ± 0.19 | 0.88 ± 0.15 | 0.84 ± 0.1 [symbol] |
| EGFR (mL/min/1.7) | 102.77 ± 18.12 | 101.25 ± 16.74 | 10 [symbol] .76 ± 15.33 | 106.61 ± 13.15 |
| BUN/Creatinine | 13.94 ± 2.93 | 13.70 ± 3.40 | 14.89 ± 4.23 | 14.37 ± 4.25 |
| Sodium (mmol/L) | 140.21 ± 2.15 | 140.12 ± 2.55 | 139.60 ± 1.79 | 139.34 ± 2.14 |
| Potassium (mmol/L) | 4.39 ± 0.33 | 4.49 ± 0.44 | 4.64 ± 0.52 | 4.36 ± 0.59 |
| Chloride (mmol/L) | 102.64 ± 2.03 | 102.33 ± 2.03 | 102.00 ± 2.10 | 102.31 ± 2.19 |
| Carbon Dioxide (mmol/L) | 22.82 ± 1.89 | 22.03 ± 1.78 | 22.31 ± 2.26 | 22.51 ± 2.42 |
| Calcium (mg/dL) | 9.52 ± 0.39 | 9.54 ± 0.33 | 9.57 ± 0.34 | 9.47 ± 0.33 |
| Total Protein (g/dL) | 3.29 ± 0.39 | 7.33 ± 0.36 | 7.31 ± 0.42 | 7.17 ± 0.16 |
| Albumin (g/dL) | 4.62 ± 0.29 | 4.6 [symbol] ± 0.25 | 4.75 ± 0.32 | 4.70 ± 0.29 |
| Globulin (g/dL) | 2.66 ± 0.31 | 2.63 ± 0.34 | 2.56 ± 0.30 | 2.47 ± 0.30 |
| A/G Ratio | 1.76 ± 0.23 | 1.79 ± 0.27 | 1.88 ± 0.25 | 1.94 ± 0.32 |
| Bilirubin (mg/dL) | 0.60 ± 0.45 | 0.39 ± 0.8 | 0.37 ± 0.32 | 0.56 ± 0.33 |
| Alkaline | 64.0 [symbol] ± 15.92 | 67. [symbol] ± 16.08 | 69.83 ± 24.41 | 68.09 ± 22.08 |
| Phosphatase (IU/L) | | | | |
| AST (IU/L) | 21.06 ± 7.52 | 22. [symbol] 4 ± 7.17 | 27.29 ± 15.80 | 23.17 ± 8.17 |
| ALT (IU/L) | 19.67 ± 12.20 | 19.21 ± 9.34 | 19.89 ± 9.74 | 19.89 ± 14. [symbol] |
| | Solarplast ® | | Placebo | |
| | Smoker | | Smoker | |
| | Pre | Post | Pre | Post |
| Glucose (mg/dL) | 91.50 ± 4.84 | 94.38 ± 4.81 | 92.88 ± 5.49 | 91.63 ± 6.48 |
| BUN (mg/dL) | 11.88 ± 2.70 | 11. [symbol] ± 4.39 | 12.13 ± [symbol] .72 | 10.75 ± 2.38 |
| Creatinine (mg/dL) | 0.74 ± 0.07 | 0.73 ± 0.09 | 0.85 ± 0.19 | 0.84 ± 0.16 |
| EGFR (mL/min/1.7) | 116.50 ± 13.84 | 116.29 ± 14.04 | 108.00 ± 14.20 | 104.63 ± 9.30 |
| BUN/Creatinine | 15.88 ± 3.31 | 16.63 ± 6.07 | 14.36 ± 4.21 | 12.88 ± 3.14 |
| Sodium (mmol/L) | 139.25 ± 1.58 | 139.13 ± 2.10 | 140.50 ± 1.20 | 141.13 ± 1.73 |
| Potassium (mmol/L) | 4.54 ± 0.38 | 4.46 ± 0.27 | 4.23 ± 0.40 | 4.28 ± 0.20 |
| Chloride (mmol/L) | 102.75 ± 1.28 | 102.63 ± 1.51 | 103.13 ± 2.03 | 102.38 ± 1.81 |
| Carbon Dioxide (mmol/L) | 21.63 ± 1.06 | 22.63 ± 1.80 | 22.75 ± 1.67 | 26.78 ± 2.38 |
| Calcium (mg/dL) | 9.40 ± 0.27 | 9. [symbol] ± 0.6 [symbol] | 9.36 ± 0. [symbol] | 9.48 ± 0.36 |
| Total Protein (g/dL) | 7.26 ± 0.37 | 7.35 ± 0.38 | 7.34 ± 0.34 | 7.33 ± 0.34 |
| Albumin (g/dL) | 4.38 ± 0.2 [symbol] | 4.66 ± 0.46 | 4.69 ± 0.32 | 4.64 ± 0.37 |
| Globulin (g/dL) | 2.69 ± 0.43 | 2. [symbol] ± 0.52 | 2. [symbol] ± 0.48 | 2.56 ± 0.47 |
| A/G Ratio | 1.73 ± 0.3 [symbol] | 1.96 ± 0.67 | 1.84 ± 0.41 | 1.88 ± 0.33 |
| Bilirubin (mg/dL) | 0.41 ± 0.13 | 0.40 ± 0. [symbol] | 0. [symbol] ± 0.33 | 0.44 ± 0.27 |
| Alkaline | 72.25 ± 15.68 | 74.13 ± 14.04 | 71.25 ± 20.30 | 73.50 ± 20.03 |
| Phosphatase (IU/L) | | | | |
| AST (IU/L) | 20.38 ± 3.26 | 20.63 ± 8.52 | 20.50 ± 10.21 | 23.25 ± 7.99 |
| ALT (IU/L) | 13.50 ± 10.78 | 15.00 ± 7.25 | 23.13 ± 14.54 | 23.63 ± 13.83 |

[symbol]

indicates data missing or illegible when filed

Oxidative Stress

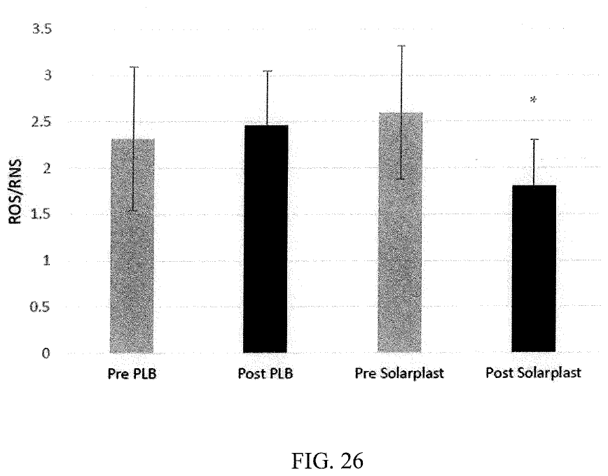

There were no significant changes found for ROS/RNS or GSH/GSSG ratio for non-smokers. A group x time interaction effect was found for ROS/RNS (p=0.046), with a significant decrease at post (p=0.031) for SOLARPLAST® supplementation (FIG. 26). No changes in GSH/GSSH were detected. Glutathione/di-glutathione assays were performed using the standard method.

Inflammation

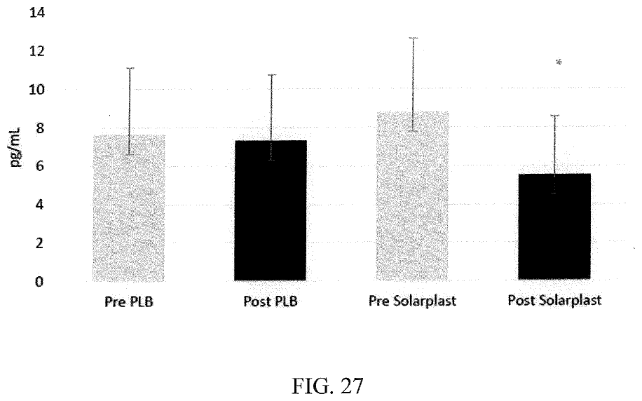

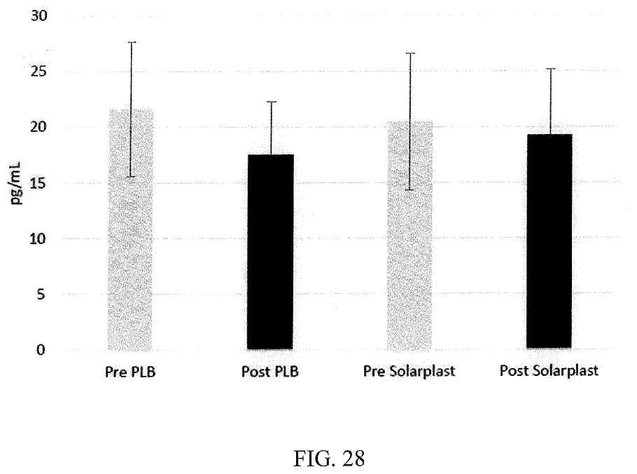

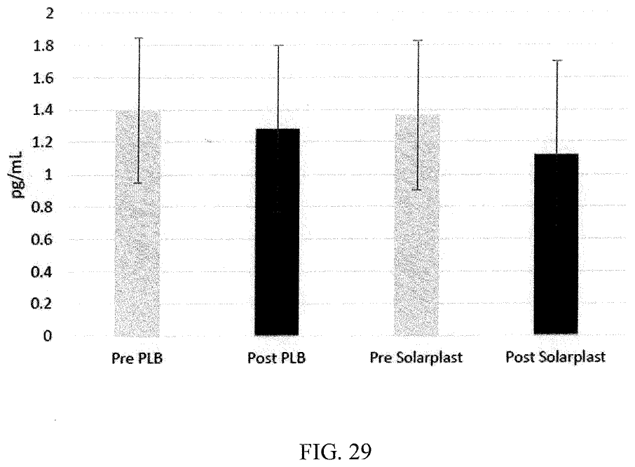

In the non-smokers group, a main effect for time was noted for IL-4 (p=0.016) and IL-6 (p=0.038) as both groups (placebo and SOLARPLAST®) experienced a decrease. No interaction results were found for IL-4 and IL-6. There was a main effect for time (decreases in both groups) and group x time interaction (p<0.001) effect for TNF-α, with significant differences at post (lower) for SOLARPLAST®, supplemented individuals (p=0.024) (FIG. 27). For inclusivity, FIGS. 28 and 29 detail results for IL-4 and IL-6 responses of non-smokers.

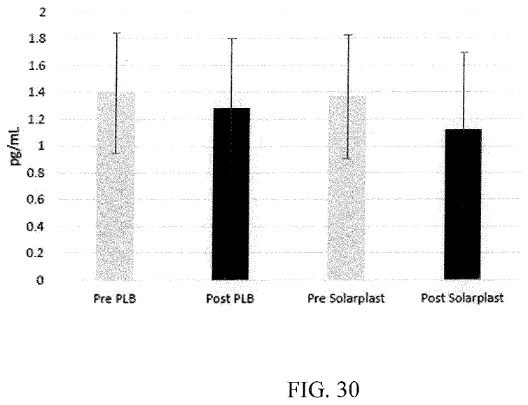

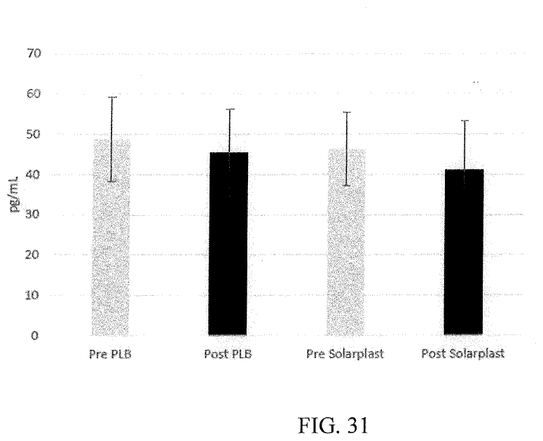

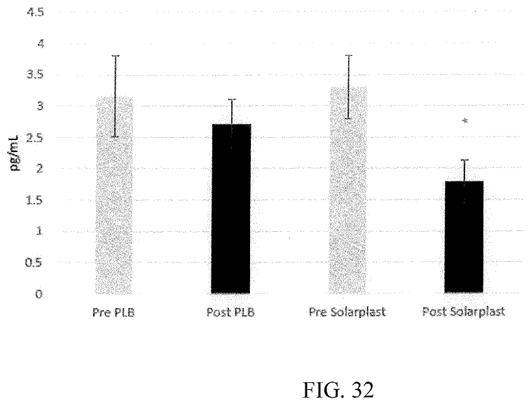

In the smokers group, there was a main effect for time for TNF-α (p=0.003), as both groups decreased (FIG. 30). No changes for IL-4 were detected (FIG. 31). A main effect for time (decrease in both groups) and a group x time interaction in smokers for IL-6 was noticed, where post hoc analysis indicated a significant decrease at post for SOLARPLAST® (p<0.001) (FIG. 32).

Skin Questionnaire (SKN-VAS)

No significant changes were detected for dryness, flushing, inconsistency of make-up, itching of the skin, eczema, body wrinkles, course skin, skin softness, elasticity, and glossiness of skin were found for non-smokers. A main effect for time was noted for flushing (p=0.01) (decreases in both groups) and overall complexion (p=0.05) (improvements in both groups), while a trend was noted for face wrinkles (p=0.052) (decreases in both groups) (Table 5).

No significant changes were found for dryness, flushing, inconsistency of make-up, itching of the skin, eczema, body wrinkles, course skin, skin softness, elasticity, glossiness of skin, face wrinkles, and overall complexion was found for non-smokers (Table 5).

Table 5 shows Skin visual analog results for smokers and non-smokers following 45 days of SOLARPLAST® or placebo supplementation, including SOLARPLAST® non-smoker (females n=22; males n=12); Placebo non-smoker (females n=16; males n=18); SOLARPLAST® smoker (females n=5; males n=3); Placebo smoker (females n=5; males n=3).

TABLE 5

| | Solarplast ® | | Placebo | | Solarplast ® | | Placebo | |
|---|---|---|---|---|---|---|---|---|
| | Non-Smoker | | Non-Smoker | | Smoker | | Smoker | |
| | Pre | Post | Pre | Post | Pre | Post | Pre | Post |
| Dryness | 1.27 ± 1.33 | 0.92 ± 1.16 | 1.71 ± 1.10 | 1.63 ± 1.26 | 1.19 ± 1.73 | 0.81 ± 1.13 | 1.81 ± 1.07 | 1.56 ± 0.82 |
| Flushing | 0.65 ± 1.19 | 0.09 ± 0.29* | 0.83 ± 1.04 | 0.71 ± 0.96* | 0.63 ± 1.19 | 0.19 ± 0.53 | 1. [symbol] 0 ± 1.85 | 1.13 ± 1.25 |
| Inconsistency | 0.34 ± 0.87 | 0.24 ± 0.51 | 0.50 ± 0.99 | 0.71 ± 1.12 | 0.17 ± 0.41 | 0.50 ± 1.22 | 0.80 ± 1.79 | 1.17 ± 1.60 |
| with Makeup | | | | | | | | |
| (females only) | | | | | | | | |
| Itching of | 0.65 ± 1.14 | 0.33 ± 0.92 | 0.61 ± 0.97 | 0.64 ± 1.10 | 0.50 ± 1.07 | 0.38 ± 0.74 | 0.75 ± 1.16 | 0.50 ± 0.76 |
| Skin | | | | | | | | |
| Eczema | 0.67 ± 1.24 | 0.39 ± 0.91 | 0.34 ± 0.84 | 0.33 ± . [symbol] 3 | 0.00 ± 0.00 | 0.00 ± 0.00 | 0.00 ± 0.00 | 0.25 ± 0.46 |
| Wrinkles (Body) | 0.15 ± 0.51 | 0.06 ± 0.24 | 0.23 ± 0.55 | 0.29 ± 0.57 | 0.25 ± 0.46 | 0.13 ± 0.35 | 0.50 ± 1.07 | 0.63 ± 10.6 |
| Wrinkles (Face) | 0.24 ± 0.50 | 0.12 ± 0.33 | 0.49 ± 0.78 | 0.40 ± 0.69 | 0.50 ± 0.76 | 0.16 ± 0.35 | 0.88 ± 1.46 | 0.88 ± 1.13 |
| Coarse Skin | 0.32 ± 1.00 | 0.36 ± 0.93 | 0.43 ± 0.88 | 0.47 ± 0.98 | 0.00 ± 0.00 | 0.00 ± 0.00 | 0.38 ± 0.74 | 0.25 ± 0.46 |
| Softness of | 3.36 ± 0.90 | 3.65 ± 1.12 | 3.27 ± 1.16 | 3.06 ± 1.28 | 3.06 ± 1.43 | 2.94 ± 2.46 | 3.31 ± 1.03 | 2.69 ± 0.96 |
| Skin | | | | | | | | |
| Elasticity of | 3.39 ± 1.34 | 3.80 ± 1.27 | 3.30 ± 1.53 | 3.13 ± 1.46 | 2.88 ± 1.64 | 3.25 ± 2.19 | 3.13 ± 1.46 | 2.25 ± 1.98 |
| Skin | | | | | | | | |
| Glossy Skin | 2.09 ± 1.48 | 2.33 ± 1.63 | 2.06 ± 1.43 | 2.01 ± 1.25 | 1.13 ± 1.36 | 2.00 ± 1.69 | 2.00 ± 1.69 | 1.38 ± 1.51 |
| Overall | 3.71 ± 0.93 | 4.06 ± 0.86** | 3.17 ± 1.02 | 3.31 ± 1.10** | 3.50 ± 0.93 | 3.50 ± 1.93 | 3.50 ± 1.07 | 2.8 [symbol] ± 1.36 |
| Complexion | | | | | | | | |
| of Skin | | | | | | | | |

*A main effect for time was noted for flushing (p = 0.01) (decreases in both groups); and
**overall complexion (p = 0.05) (improvements in both groups). [symbol]
indicates data missing or illegible when filed

Anti-Aging QOL Common Questionnaire: Physical

In non-smokers, no significant findings were noted for tired eyes, blurry eyes, stiff shoulders (though a trend was noted for an interaction; p=0.06), palpitations, shortness of breath, tendency to gain weight, weight loss, lethargy, no feeling of good health, thirst, anorexia, early satiety, epigastric, liable to catch a cold, cough and sputum, diarrhea, constipation, gray hair, dizziness, tinnitus, arthralgia, edematous, frequent urination, hot flash, and cold skin. A main effect for time was noted for eye pain (p=0.028) (decreases in both groups), headache (p=0.003) (decreases in both groups), lumbago (p=0.010) (decreases in both groups), and sweat (p=0.045) (decreases in both groups) was noted in non-smokers. A group x time interaction was noted for muscular pain (p=0.031), where significant decreases were found at post (p=0.026). A main effect for time (p=0.009) (decrease in skin problems in both groups) and group x time interaction (p=0.002) was found for skin problems, where significant differences at post indicated decreases in skin problems for those who supplemented with SOLARPLAST® (p=0.010).

In smokers, no significant findings were noted for tired eyes, blurry eyes, eye pain, muscular pain, palpitations, shortness of breath, tendency to gain weight, weight loss, lethargy, no feeling of good health, skin problems, anorexia, early satiety, epigastric, liable to catch a cold, cough and sputum, diarrhea, constipation, gray hair, hair loss, headache, dizziness, tinnitus, lumbago, arthralgia, edematous, sweat, frequent urination, hot flash, and cold skin. A main effect for time for decreases in stiff shoulders (p=0.009) and decreases in thirst (p=0.028) was noted in smokers.

Table 6 shows select representative data that indicated significant differences from the physical aspect of the AAQOL survey.

TABLE 6

| | Solarplast ® | | Placebo | |
|---|---|---|---|---|
| | Non-Smoker | | Non-Smoker | |
| | Pre | Post | Pre | Post |
| Muscular | 1.63 ± 0.74 | 1.00 ± 0.00* | 1.75 ± 0.71 | 1.63 ± 0.74 |
| pain | | | | |
| Skin | 1.67 ± 1.14 | 1.25 ± 0.70** | 1.40 ± 0.88 | 1.53 ± 1.01 |
| problems | | | | |

Two-way ANOVA indicated group by time interaction
*p = 0.026 ;
**= p = 0.010.

Anti-Aging QOL Common Questionnaire: Mental

In non-smokers, no significant findings were noted for easily angered, loss of motivation, no feelings of happiness, nothing to look forward to in life, daily life is enjoyable, reluctance to talk with others, depressed, feeling of usefulness, pessimism, lapse in memory, inability to solve problems, inability to make judgments, and vague feelings of fear. A main effect for time was noted for loss of confidence (p=0.006) (decreased, which is a positive response). Similarly, a main effect for decreases in inability to concentrate (which is a positive finding) (p=0.017) and decreases in a sense of tension (p=0.01) (also a positive finding) were found. A main effect for time (p=0.001) (decreased in both groups) and group-by-time interaction was found for feelings of anxiety for no specific reason, where significant decreases were noticed for SOLARPLAST® at post (p=0.002) (a positive finding). Lastly, a main effect for time was found for irritability (p=0.025) (decreases in both groups), as well as a group x time interaction effect, where irritability was decreased at post (p=0.025) (Table 7) for SOLARPLAST®.

Table 7 shows select representative data that indicated significant differences from the mental aspect of the AAQOL survey for non-smokers.

TABLE 7

| | Solarplast ® | | Placebo | |
|---|---|---|---|---|
| | Non-Smoker | | Non-Smoker | |
| | Pre | Post | Pre | Post |
| Anxiety for | 1.82 ± 1.19 | 1.18 ± 0.47* | 1.63 ± 1.21 | 1.51 ± 0.89 |
| no specific | | | | |
| reason | | | | |
| Irritability | 1.73 ± 0.88 | 1.27 ± 0.57** | 1.63 ± 0.84 | 1.63 ± 0.81 |

Two-way ANOVA indicated group by time interaction
*p = 0.002;
**= p = 0.025

In smokers, a group x time interaction effect was found for depression, where depression was decreased in smokers at post (p=0.029) (Table 8). No main effect or interaction was noted for easily angered, no feelings of happiness, nothing to look forward to in life, daily life is enjoyable, loss of confidence, reluctance to talk with others, lapse in memory, inability to solve problems, inability to make judgments, a sense of tension, feelings of anxiety for no specific reason, and vague feelings of fear. A main effect for time was noted for irritability (p=0.013) (decreased, which is a positive response); however, no interaction was found. A main effect for time was noted for loss of motivation (p=0.018), feeling of usefulness (p=0.005), pessimism (p=0.049), inability to concentrate (p=0.017).

Table 8 shows select representative data that indicated significant differences from the mental aspect of the AAQOL survey for smokers.

TABLE 8

| | Solarplast ® | | Placebo | |
|---|---|---|---|---|
| | Smoker | | Smoker | |
| | Pre | Post | Pre | Post |
| Depression | 2.13 ± 1.13 | 1.13 ± 0.35 | 1.25 ± 0.46 | 1.63 ± 0.52 |

Two-way ANOVA indicated group by time interaction
*p = 0.029.

### Discussion

It is believed that the above study is the first randomized, placebo-controlled trial conducted on the enzymatically enhanced spinach supplement, SOLARPLAST®. No participants reported adverse events associated with the supplement during the 45-day, daily consumption period. Further, clinical hematological biomarkers related with health were examined, with no abnormal results reported as a consequence of the 45-day supplementation period. Mean values for the comprehensive metabolic exam were all within normal clinical limits pre- and post-supplementation for both non-smokers and smokers. This is unsurprising, as the enzymatically enhanced organic spinach contains no foreign materials that would be expected to negatively impact health. It can be concluded, SOLARPLAST® is safe to consume by young, healthy adults, as well as individuals who smoke.

Due to the often inadequate consumption of fruits and vegetables, important external sources of antioxidants for humans, supplementation is warranted as routine consumption of antioxidants is proposed to reduce the risk of lifestyle-related illnesses such as diabetes, obesity, and cardiovascular disease. Per Deerland Enzymes & Probiotics, SOLARPLAST® is unique in that by using the preparative process described herein, organic spinach is enzymatically enhanced and capitalizes on protoplast (isolated plant cells that lack the rigid cellulose walls found in intact tissue) activity. Since this method removes the plant cell wall, it can enhance digestion and absorption of micronutrients which can assist in the human antioxidant defense system. It is asserted here that antioxidant power is exponentially greater due to the presence of enzymes that facilitate the glutathione pathway of antioxidant regeneration. Several studies have demonstrated the antioxidant activities of spinach, in various forms (powder, raw), to date. However, once fruits and vegetables are harvested the generation of ROS is initiated, reducing antioxidants through a plethora of reduction-oxidation reactions promotes cell proliferation and survival to maintain cellular homeostasis. Moreover, products are further compromised by storage and distribution times. The storage process during postharvest reduced the content of antioxidants such as vitamin C and the scavenging activity of flavonoids in spinach. Given SOLARPLAST® was created to provide two times the total antioxidant capacity of regular spinach, the examination of its impact on ROS/RNS and GSG/GSSH activity (i.e., increased GSG/GSSH activity is indicative of oxidative stress) was of interest.

Following 45 days of supplementation by non-smokers there was no impact of daily SOLARPLAST® supplementation on ROS/RNS (cumulative oxidative stress) or GSG/GSSH. The non-diseased status of these participants may have contributed to these results overall, as well as the age status of “young”. Participants in the non-smoking group reported engaging in exercise (SOLARPLAST®: 127.7±30.2 min/week; placebo: 135.7±32.5 min/week) throughout the study enrollment, which can promote free radicals in the short term, but increases antioxidant capacity over the long term, as well as minimal alcohol intake (which increases oxidative stress) intake per week (SOLARPLAST®: 1.1±0.7; placebo: 0.9±0.5 drinks per week).

ROS/RNS changed significantly in smokers who supplemented with SOLARPLAST®. Specifically, ROS/RNS significantly decreased, while GSH/GSSG did not change following 45 days of supplementation. It is thought that smoking causes increased oxidative stress for a host of reasons, including direct damage by radical species and the inflammatory response caused by cigarette smoking. More specifically, two focal phases have been identified in cigarette smoke—the tar phase and gas phase. The two phases are composed of stimulating significant free radical and non-radical oxidant production. For example, hydroxyl and superoxide radicals, as well as peroxyl initiate oxidative damage in the form of lipid peroxidation. Peroxyl radicals and reactive nitrogen species cause direct damage stimulating lipid peroxidation and oxidation of proteins and DNA bases and aldehydes in smoke can lessen GSH (reduced glutathione). The outcomes from 45 days of SOLARPLAST® supplementation in smokers are promising. Smoking cessation would be the ideal preventative step, however, not all individuals will quit or reduce their cigarette usage, thus providing some protection from systemic oxidative stress via daily SOLARPLAST® consumption appears to be a safe option for smokers.

Interleukin-6 (IL-6) is a multifunctional cytokine that participates in the inflammatory and immune responses. Its immunological activities include B cell differentiation and stimulation of IgG secretion, T cell differentiation and growth, and cytotoxic T cell differentiation. IL-6 is produced by activated monocytes, macrophages, endothelial cells, fibroblasts, keratinocytes, and activated T and B cells in response to induction by a variety of stimuli which include other cytokines. The functions of TNF-α are mediated through its two main receptors: tumor necrosis factor receptor 1 (TNFR1) and tumor necrosis factor receptor 2 (TNFR2). Activation of TNFR1 is known to initiate inflammatory, apoptotic, and degenerative cascades, whereas TNF-α signaling through TNFR2 is anti-inflammatory and cytoprotective, resulting in the induction of proliferation, differentiation, angiogenesis, and tissue repair. TNF-α can induce secretion of IL-6 by keratinocytes, macrophages, and endothelial cells.

Results showed non-smokers experienced significant reductions in TNF-α. No changes in IL-4 or IL-6 were detected. The reduction of TNF-α in the healthy participant cohort is interesting, as these young individuals were non-diseased individuals who had, on average, a normal body fat percentage (obesity is associated with higher serum TNF-α levels) and normal levels of TNF-α. Research in clinical populations has demonstrated interplay between glutathione deficiency and increased TNF-α concentrations. Perhaps the antioxidants contained in SOLARPLAST® purported to improve glutathione antioxidant pathway activity played a role in the reduction, but more research is required to understand the relationship.

Analysis of IL-6 in smokers who consumed SOLARPLAST® indicated a significant reduction at the 45-day time-point. Smoking is well known to enhance the production of pro-inflammatory molecules by numerous cell types as well as elevated, systemic inflammatory biomarkers (e.g., CRP, IL-6, TNF-α). Various inflammatory stimuli such as excessive ROS/RNS produced in the process of oxidative metabolism and some natural or artificial chemicals (e.g., smoking) have been stated to initiate the inflammatory process resulting in synthesis and exudation of proinflammatory cytokines. Perhaps the reduction in ROS/RNS played a role in the reduction in IL-6 production. The reduction is particularly important for smokers as IL-6 is highly associated with an increased risk of asthma and other smoking-related diseases. It should be noted that not all investigations have found increases in basal IL-6 concentrations in smokers, rather, reductions in IL-6 compared to non-smokers. Thus, more research should be conducted on IL-6 and other cytokines, as well as their interaction with antioxidants.

Skin, physical, and mental health were also assessed via a questionnaire as part of the investigation. In non-smokers, self-reported skin health was improved in individuals who supplemented with SOLARPLAST®. This finding is interesting, as these participants were young and have not experienced “aging skin”. The cutaneous antioxidant system consists of enzymatic and non-enzymatic substances. Among enzymatic antioxidants, glutathione peroxidase (GPx), catalase (CAT) and superoxide dismutase (SOD) are a few that impact skin health. As such, SOLARPLAST® may have positively contributed to the cellular redox status of their skin; however, more research is needed on the exact mechanism. Healthy participants who supplemented with SOLARPLAST® also experienced decreased “irritability” and “anxiety for no specific reason.” A host of reasons could have contributed to these results, with which need further exploration. Interestingly, TNF-α, which was significantly reduced in these healthy participants has been associated with reduced emotional and cognitive disturbances in humans; however, these require further examination as this is often studied in more clinical populations. Also plausible is the fact many of the healthy participants were students, and thus the stress of a changing semester is a plausible cause, as well as the prevalence of the COVID-19 pandemic.

In smokers, SOLARPLAST® supplementation significantly reduced feelings of depression. Previous research indicates that smokers are more likely to report symptoms of depression than non-smokers. There is also evidence that pro-inflammatory cytokines such as C-reactive protein (CRP), interleukin-1 beta (IL-1-b), TNF-α, and IL-6 are increased in depressive disorder. Nunes, et al., compared inflammatory markers in depressed and non-depressed smokers and found that depressed smokers had higher serum CRP, IL-6, and TNF-α levels than nondepressed smokers and had worse physical health outcomes and greater work-related disability. It is thought that shared inflammatory and oxidative stress may be part of the reason for the common co-occurrence of depression and medical disorders, and reductions may reduce feelings of depression (See, Nunes, et al., “A comparison of inflammatory markers in depressed and nondepressed smokers,” Nicotine Tob. Res. (2012) 14(5):540-6). As both ROS/RNS and IL-6 decreased significantly, as well as a decrease (non-significant) in TNF-α occurred in Solarlast supplemented smokers, perhaps these were contributing factors to decreased depression feeling reporting.

### CONCLUSIONS

SOLARPLAST® appears to be effective at reducing conditions such as oxidative stress and inflammation in individuals who experience higher basal levels, smokers. Further, SOLARPLAST® may also benefit feelings of depression, which are commonly reported by smokers. Lastly, while young healthy adults may not have high basal levels of inflammation and oxidative stress, promising results were found on the impact of SOLARPLAST® on skin health and reducing inflammation.

In an embodiment, administration of SOLARPLAST® may be used to improve one or more of oxidative stress and associated symptoms thereof, inflammation and associated symptoms thereof, various skin conditions as discussed above, and various conditions associated with aging as discussed above.

Suitable dosages of SOLARPLAST® as described herein can range from about 100 mg to about 1000 mg for oral administration to human patients on a daily basis. Another suitable dosage range for SOLARPLAST® as described herein can range from about 50 mg to about 5000 mg for oral administration to human patients on a daily basis.

The compositions of the present invention may be formulated into nutraceutical or pharmaceutical oral solid dosage forms, such as tablets, capsules, powders or into liquid formulations, such as solutions and suspensions using suitable excipients.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approx. ±10%; in other embodiments the values may range in value either above or below the stated value in a range of approx. ±5%; in other embodiments the values may range in value either above or below the stated value in a range of approx. ±2%; in other embodiments the values may range in value either above or below the stated value in a range of approx. ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entireties. The present invention may be embodied in other specific forms without departing from the spirit or essential attributes thereof and, accordingly, reference should be made to the appended claims, rather than to the foregoing specification, as indicating the scope of the invention.

What is claimed is:

1. An enzymatic method for preparing protoplasts, comprising the steps of:
(a) preparing an aqueous solution including an osmotic protectant;
(b) adding plant material in an amount of about 0.1% by weight to about 50% by weight based on the total weight of the aqueous solution to produce a plant mixture;
(c) heating the plant mixture to a range of from about 30° C. to about 37° C.;
(d) treating the heated plant mixture with an aqueous solution of one or more cell-wall degrading enzymes for up to 2 hours to provide a protoplast containing mixture;
(e) cooling the protoplast containing mixture to room temperature or less than room temperature;
(f) adding a bulk density enhancing material to the protoplast containing mixture;
(g) drying the protoplast containing mixture;
(h) grinding or milling the dried protoplast containing mixture to provide protoplasts; wherein the cell membranes of the protoplasts are isolated generally intact.

2. The enzymatic method of claim 1, wherein the plant material is selected from the group consisting of kale, spinach, hemp, collard greens, mustard greens, arugula, swiss chard, bok choy leaves, and turnip greens.

3. The enzymatic method of claim 1, wherein the plant material is spinach identified as genus and species Spinacia oleracea.

4. The enzymatic method of claim 1, wherein the cell-wall degrading enzymes are selected from the group consisting of cellulase, pectinase, and xylanase.

5. The enzymatic method of claim 4, wherein the cell-wall degrading enzymes are added in an amount of about 0.1-50% by wt. based on plant material weight.

5. The enzymatic method of claim 1, wherein the osmotic protectant comprises erythritol, trehalose, sorbitol, or mixtures thereof.

6. The enzymatic method of claim 1, wherein the bulk density enhancing material is selected from organic fibers, oligosaccharides, starches, or mixtures thereof.

7. A protoplast enriched composition prepared by the method of claim 1.

8. A dietary supplement comprising a protoplast enriched composition and a nutraceutically acceptable carrier.

9. The dietary supplement of claim 8, wherein the protoplast enriched composition is derived from Spinacia oleracea.

10. The dietary supplement of claim 9 having a CAP-e bioassay value of about 4.8 to about 13 micromolar gallic acid equivalents per gram.

11. The dietary supplement of claim 8, provided in a solid form or capsule in an amount of about 100 mg to about 1000 mg.

12. A method for treating oxidative stress in a human individual, comprising the steps of:
(a) providing a protoplast containing composition; and
(b) administering to the human in need thereof a therapeutically or nutraceutically acceptable amount of the protoplast containing composition;
wherein cellular or intracellular markers of oxidative stress are reduced.

13. The method of claim 12, wherein the protoplast containing composition is administered in an amount of about 50 mg to about 5000 mg on a daily basis.

14. The method of claim 12, wherein the protoplast containing composition is administered in an amount of about 100 mg to about 1000 mg on a daily basis.

15. The method of claim 12, wherein step (b) is carried out for 45 days and ROS/RNS levels are reduced.

16. A method for treating inflammation in a human individual, comprising the steps of:
(a) providing a protoplast containing composition; and
(b) administering to the human in need thereof a therapeutically or nutraceutically acceptable amount of the protoplast containing composition;
wherein cellular or intracellular markers of inflammation are reduced.

17. The method of claim 16, wherein the protoplast containing composition is administered in an amount of about 50 mg to about 5000 mg on a daily basis.

18. The method of claim 16, wherein the protoplast containing composition is administered in an amount of about 100 mg to about 1000 mg on a daily basis.

19. The method of claim 16, wherein step (b) is carried out for 45 days and serum TNF-α levels are reduced.

20. The method of claim 16, wherein step (b) is carried out for 45 days and serum IL-6 levels are reduced.
